(19) United States
(12) Patent Application Publication
(10) Pub. No.: US 2026/0219267 A1
(43) Pub. Date: Jul. 30, 2026

(54) METHODS AND SYSTEMS FOR LABEL-FREE ANALYTE DETECTION

(71) Applicant: Pumpkinseed Technologies, Inc., Palo Alto, CA (US)

(72) Inventors: Jennifer Dionne, Palo Alto, CA (US); Nhat Vu, Palo Alto, CA (US); Jack Hu, Palo Alto, CA (US)

(21) Appl. No.: 19/146,436

(22) PCT Filed: Jan. 8, 2024
(86) PCT No.: PCT/US2024/010744

§ 371 Date: Jul. 8, 2025

Related U.S. Application Data
(60) Provisional application No. 63/479,165, filed on Jan. 9, 2023.

(51) Int. Cl. G01N 33/543; G01N 21/64; G01N 21/65

(52) U.S. Cl. CPC G01N 33/54373; G01N 21/64; G01N 21/65

(57) ABSTRACT

The present disclosure provides methods and systems for analyte detection. The system may comprise a chip. The chip may comprise an array of non-uniform features. A feature of the array of non-uniform features may comprise an electrical insulator or a semiconductor. The feature may further comprise a nanogap.

### CROSS-REFERENCE

This application claims the benefit of U.S. Provisional Application No. 63/479,165, filed Jan. 9, 2023, which is entirely incorporated herein by reference.

### BACKGROUND

Sample analysis methods and devices have enabled deep analysis of materials, especially biological materials. Analysis of non-labeled samples can provide insights into the properties of the sample and can improve acquisition speed and sample fidelity.

### SUMMARY

Recognized herein is the need for high sensitivity and high specificity analysis methods and systems. A system that can reliably and repeatably generate data (e.g., sequencing data or other identifying data) for single analytes can provide a powerful platform for diagnostics (e.g., determining a disease state of a subject), target discovery (e.g., discovering a target for a therapeutic), sample quality analysis (e.g., measuring a level of an impurity or product in a production line, etc.), or the like. Such a system can provide faster and lower cost analysis.

The methods and systems of the present disclosure can be used as such a high sensitivity and high specificity analysis platform. The arrays of the present disclosure which comprise a nanogap can provide enhanced concentration of light on a near-field regime, thereby enhancing the signal that can be generated from an analyte within the gap and reducing the amount of analyte that is needed for analysis. Further, such a system can control far-field scattering of light, further enhancing efficiency and reducing system noise.

Additionally, use of a resonator comprising a nanogap can enable label-free analyte analysis (e.g., detection, identification, identification of modifications to the analyte (e.g., post-translational modification, etc.)). For example, Raman spectroscopy can provide information related to the entire analyte in a single spectrum due to Raman's probing of the various vibrational states of the analyte. This full analyte data set can then be analyzed to provide not only the identity of the analyte, but also data related to any perturbations of the analyte from a reference analyte (e.g., modifications, mutations, presence or absence of a cofactor, etc.). In this way, analytes including polypeptides, proteins, small molecules, and even cells or multicellular organisms, can be analyzed. Not only can single analytes be probed, but interactions like protein-protein interactions, antibody-drug interactions, biomolecule-therapeutic interactions, and the like can be probed.

A plurality of resonators can be fabricated on a same chip and, due in part to the relatively small size of the resonators, can enable parallelized analysis of many analytes from a sample simultaneously. For example, different resonators can be functionalized to bind different portions of a sample mixture, and wide field illumination and detection schemes can be used to probe the resonators and thus gather data related to a number of analytes at the same time. In some cases, the sample can be purified on chip (e.g., using features of the chip such as a plurality of non-uniform features to filter the sample), off-chip (e.g., an off-chip liquid or solid chromatography system can be utilized to filter the analytes from the rest of the sample before the analytes are introduced to the chip), or a combination thereof. In some cases, the sample can be introduced to the chip without purification, permitting analysis of many different analytes without additional processing operations.

In an aspect, the present disclosure provides a method of determining an identity of an analyte, comprising: (a) providing a system comprising: (i) chip comprising a resonator comprising a nanogap, wherein the nanogap comprises the analyte, (ii) a light source, and (iii) a detector; (b) illuminating the resonator using light from the light source, wherein the resonator concentrates a field of the light into the nanogap; (c) detecting, using the detector, an emission light generated by an interaction of the field with the analyte; and (d) processing the emission light or a derivative thereof to determine the identity of the analyte.

In some embodiments, the resonator comprises an array of non-uniform features. In some embodiments, a feature of the array of non-uniform features comprises the nanogap. In some embodiments, (d) comprises using the emission light to generate a spectrum, and processing the spectrum to determine the identity. In some embodiments, the interaction is a Raman interaction. In some embodiments, the analyte is a biological analyte. In some embodiments, the biological analyte is a protein, polypeptide, or peptide. In some embodiments, the analyte is a chemical analyte. In some embodiments, the analyte does not comprise a label or have a label bound thereto. In some embodiments, the light source is a laser light source.

In another aspect, the present disclosure provides a chip for analyzing a biological sample, the chip comprising an array of non-uniform features, wherein a feature of the array of non-uniform features comprises an electrical insulator or a semiconductor, wherein the feature comprises a nanogap.

In some embodiments, the nanogap is configured to concentrate an incident light. In some embodiments, the incident light is an incident laser. In some embodiments, the incident laser is at a wavelength of at least about 400 nanometers (nm) to at least about 1800 nanometers (nm). In some embodiments, the incident light is a light emitting diode (LED) light. In some embodiments, the incident light is a lamp. In some embodiments, the nanogap comprises a binding moiety that is specific or non-specific for the analyte. In some embodiments, the nanogap comprises a binding moiety with binding specificity for the analyte. In some embodiments, the biological sample comprises two or more components. In some embodiments, the chip further comprises an additional array comprising non-uniform features configured to filter the two or more components according to size, charge, or binding affinity. In some embodiments, the non-uniform pillars of the additional array are interspersed with a plurality of electrodes or functionalized features configured to filter the two or more components according to charge or size. In some embodiments, the functionalized feature comprises a functionalized oxide surface. In some embodiments, the biological sample is a tissue sample. In some embodiments, the biological sample is a single cell. In some embodiments, the analyte is a polynucleotide. In some embodiments, the analyte is a protein, polypeptide, or peptide. In some embodiments, the analyte is a metabolite. In some embodiments, the biological sample is an organism. In some embodiments, the organism is a bacterium. In some embodiments, the organism is a virus. In some embodiments, the biological sample comprises a cell fragment. In some embodiments, at least one feature of the array has a height of at least about 50 nanometers (nm) to at least about 1000 nanometers (nm). In some embodiments, at least one feature of the array has a width of at least about 50 nanometers (nm) to at least about 500 nanometers (nm). In some embodiments, at least one feature of the array has a length of at least about 50 nanometers (nm) to at least about 2000 nanometers (nm). In some embodiments, the distance between the non-uniform features is about 50 nanometers (nm) to about 1000 nanometers (nm). In some embodiments, the chip further comprises a first subset of the array of non-uniform features and a second subset of the array of non-uniform features, wherein the first subset and the second subset are adjacent to one another. In some embodiments, the first subset of the array of non-uniform features is separated from the second subset of the array of non-uniform features by a distance of at least about 5 nanometers (nm) to at least about 3000 nanometers (nm). In some embodiments, the first subset of the array of non-uniform features is separated from the second subset of the array of non-uniform features by a distance of at least about 2000 nanometers (nm). In some embodiments, the first subset of the array of non-uniform features is separated from the second subset of the array of non-uniform features by a distance of less than about 1000 nanometers (nm). In some embodiments, the first subset of the array of non-uniform features is parallel to the second subset of the array of non-uniform features. In some embodiments, the first subset of the array of non-uniform features is separated from the second subset of the array of non-uniform features by one or more dielectric fins. In some embodiments, the first subset of the array of non-uniform features is not parallel to the second subset of the array of non-uniform features. In some embodiments, two or more non-uniform features of the array comprise a nanogap. In some embodiments, three or more non-uniform features of the array comprise a nanogap. In some embodiments, each the feature of the array comprises a nanogap. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the non-uniform features of the array comprise one or more materials from the group consisting of silicon, silicon nitride, aluminum nitride, titanium dioxide, silicon dioxide, gallium nitride, hafnium oxide, germanium, and silicon carbide. In some embodiments, the chip is provided on a substrate. In some embodiments, the substrate comprises one or more materials from the group consisting of germanium aluminum oxide, silicon dioxide, fused silica, silicon dioxide on silicon, silicon, silicon nitride, gallium nitride, calcium fluoride, and beryllium oxide. In some embodiments, two or more of the non-uniform features of the array are parallel to one another. In some embodiments, two or more of the non-uniform features of the array are nonparallel to one another. In some embodiments, the non-uniform features of the array are rectangular. In some embodiments, the non-uniform features of array are rounded. In some embodiments, the non-uniform features of the array are arranged in a periodic configuration. In some embodiments, the non-uniform features of the array are arranged in a nonperiodic configuration. In some embodiments, a non-uniform feature of the array is a photonic crystal mirror. In some embodiments, the array has a quality factor of at least about 100. In some embodiments, the array has a mode volume of at most about 800 nanometers.

In another aspect, the present disclosure provides a method of detecting or identifying an analyte in a biological sample, comprising: (a) providing the biological sample on a chip comprising a resonator comprising a nanogap; (b) exposing the chip to a first light from a light source, such that the first light interacts with the resonator and is further concentrated in the nanogap; (c) detecting a second light from the resonator subsequent to the resonator being exposed to the first light; and (d) using the second light to detect or identify the analyte.

In some embodiments, the resonator comprises an array comprising a plurality of non-uniform features, wherein a feature of the plurality of non-uniform features comprises the nanogap. In some embodiments, the second light yields an infrared scattering signature associated with the analyte. In some embodiments, the second light yields a vibrational scattering signature associated with the analyte. In some embodiments, the vibrational scattering signature is a Raman spectrum. In some embodiments, the second light yields autofluorescence associated with the analyte. In some embodiments, the light source of the first light is integrated with the chip. In some embodiments, the light source of the first light is not integrated with the chip. In some embodiments, the a light source of the second light is integrated with the chip. In some embodiments, the light source of the second light is not integrated with the chip. In some embodiments, the method further comprises detecting the presence or absence of the analyte. In some embodiments, the nanogap comprises a binding moiety with binding specificity for the analyte. In some embodiments, the nanogap comprises a binding moiety with binding specificity for the analyte. In some embodiments, the nanogap is configured to concentrate an incident light. In some embodiments, the first light is an incident laser. In some embodiments, the incident laser is at a wavelength of at least about 400 nanometers (nm) to at least about 1800 nanometers (nm). In some embodiments, the first light is a light emitting diode (LED) light. In some embodiments, the first light is a lamp. In some embodiments, the method further comprises providing a detector. In some embodiments, the method further comprises using the detector to scan wavelengths in a detector plane. In some embodiments, the method further comprises using super resolution imaging to image at least a portion of the chip. In some embodiments, the super resolution imaging is structured illumination microscopy (SIM). In some embodiments, the super resolution imaging is entropy based super resolution imaging (ESI). In some embodiments, the super resolution imaging is stochastic optical reconstruction microscopy (STORM). In some embodiments, the super resolution imaging is super resolution optical fluctuation imaging (SOFI). In some embodiments, the super resolution imaging is stimulated emission depletion microscopy (STED). In some embodiments, the method further comprises producing one or more hyperspectral images. In some embodiments, each of the one or more hyperspectral images represents a distinct Raman signature. In some embodiments, the method further comprises collecting data from the one or more hyperspectral images. In some embodiments, the method further comprises developing a machine learning model. In some embodiments, a computing system is configured to execute the machine learning model. In some embodiments, the computing system comprises a neural network. In some embodiments, the neural network is a convolutional neural network (CNN). In some embodiments, the biological sample comprises two or more components. In some embodiments, the method further comprises an additional array of non-uniform features configured to filter the two or more components according to size, charge, or binding affinity. In some embodiments, the additional array of non-uniform features is interspersed with a plurality of electrodes or functionalized features configured to filter the two or more components according to charge. In some embodiments, the functionalized feature comprises a functionalized oxide surface. In some embodiments, the biological sample is a tissue sample. In some embodiments, the biological sample is a single cell. In some embodiments, the analyte is a polynucleotide. In some embodiments, the analyte is a protein, polypeptide, or peptide. In some embodiments, the analyte is a metabolite. In some cases, the metabolite is a lipid. In some cases, the metabolite is a lipid. In some embodiments, the biological sample is an organism. In some embodiments, the organism is a bacterium. In some embodiments, the organism is a virus. In some embodiments, the biological sample comprises a cell fragment. In some embodiments, at least one feature of the resonator has a height of at least about 50 nanometers (nm) to at least about 1000 nanometers (nm). In some embodiments, at least one feature of the resonator has a width of at least about 50 nanometers (nm) to at least about 500 nanometers (nm). In some embodiments, at least one feature of the resonator has a length of at least about 50 nanometers (nm) to at least about 2000 nanometers (nm). In some embodiments, the distance between the non-uniform features of the array is about 50 nanometers (nm) to about 1000 nanometers (nm). In some embodiments, the method further comprises a first subset of the array of non-uniform features and a second subset of the array of non-uniform features, wherein the first subset and the second subset are adjacent to one another. In some embodiments, the first subset of the array of non-uniform features is separated from the second subset of the array of non-uniform features by a distance of at least about 5 nanometers (nm) to at most about 3000 nanometers (nm). In some embodiments, the first subset of the array of non-uniform features is separated from the second subset of the array of non-uniform features by a distance of at least about 2000 nanometers (nm). In some embodiments, the first subset of the array of non-uniform features is separated from the second subset of the array of non-uniform features by a distance of less than about 1000 nanometers (nm). In some embodiments, the first subset of the array of non-uniform features is parallel to the second subset of the array of non-uniform features. In some embodiments, the first subset of the array of non-uniform features is separated from the second subset of the array of non-uniform features by one or more dielectric fins. In some embodiments, the first subset of the array of non-uniform features is not parallel to the second subset of the array of non-uniform features. In some embodiments, two or more non-uniform features of the resonator comprise a nanogap. In some embodiments, three or more non-uniform features of the resonator comprise a nanogap. In some embodiments, each feature of the resonator comprises a nanogap. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the non-uniform features of the array comprise one or more materials from the group consisting of silicon, silicon nitride, aluminum nitride, titanium dioxide, silicon dioxide, gallium nitride, hafnium oxide, germanium, and silicon carbide. In some embodiments, the chip is provided on a substrate. In some embodiments, the substrate comprises one or more materials from the group consisting of germanium, aluminum oxide, silicon dioxide, fused silica, silicon dioxide on silicon, silicon, silicon nitride, gallium nitride, calcium fluoride, and beryllium oxide. In some embodiments, two or more of the non-uniform features of the array are parallel to one another. In some embodiments, two or more of the non-uniform features of the array are nonparallel to one another. In some embodiments, the non-uniform features of the array are rectangular. In some embodiments, the non-uniform features of the array are rounded. In some embodiments, the non-uniform features of the array are arranged in a periodic configuration. In some embodiments, the non-uniform features of the array are arranged in a nonperiodic configuration. In some embodiments, a non-uniform feature of the array is a photonic crystal mirror.

In another aspect, the present disclosure provides a label-free method for detecting an analyte, comprising providing a chip comprising the analyte, wherein the analyte is label-free, and using light to detect an identity of the analyte at a sensitivity of at least 80%, a specificity of at least 80%, or an accuracy of at least 80%.

In another aspect, the present disclosure provides a chip for processing a biological sample comprising one or more components, the chip comprising an array of non-uniform features configured to filter the one or more components according to size, wherein a feature of the array of non-uniform features comprises an electrical insulator or a semiconductor, and wherein the array of non-uniform features are interspersed with a plurality of electrodes or functionalized features configured to filter the one or more components according to size or charge.

In some embodiments, the biological sample is a tissue sample. In some embodiments, the biological sample is a single cell. In some embodiments, the biological sample comprises a polynucleotide. In some embodiments, the biological sample comprises a protein, polypeptide, or peptide. In some embodiments, the biological sample comprises a metabolite. In some embodiments, the biological sample is an organism. In some embodiments, the organism is a bacterium. In some embodiments, the organism is a virus. In some embodiments, the biological sample comprises a cell fragment. In some embodiments, the chip comprises an additional array of non-uniform features, wherein at least one feature of the additional array comprises a nanogap. In some embodiments, the nanogap is configured to concentrate an incident light. In some embodiments, the incident light is a laser. In some embodiments, the laser is at a wavelength of at least about 400 nanometers (nm) to at least about 1800 nanometers (nm). In some embodiments, the incident light is a light emitting diode (LED) light. In some embodiments, the incident light is a lamp. In some embodiments, the nanogap comprises a binding moiety that is specific or non-specific for the analyte. In some embodiments, the nanogap comprises a binding moiety with binding specificity for the analyte. In some embodiments, at least one non-uniform feature of the additional array has a height of at least about 50 nanometers (nm) to at least about 1000 nanometers (nm). In some embodiments, at least one non-uniform feature of the additional array has a width of at least about 50 nanometers (nm) to at least about 500 nanometers (nm). In some embodiments, at least one non-uniform feature of the additional array has a length of at least about 50 nanometers (nm) to at least about 2000 nanometers (nm). In some embodiments, the distance between the non-uniform features of the additional array is about 50 nanometers (nm) to about 1000 nanometers (nm). In some embodiments, the chip comprises a first subset of the additional array of non-uniform features and a second subset of the additional array of non-uniform features, wherein the first subset and the second subset are adjacent to one another. In some embodiments, the first subset of the additional array of non-uniform features is separated from the second subset of the additional array of non-uniform features by a distance of at least about 5 nanometers (nm) to at most about 3000 nanometers (nm). In some embodiments, the first subset of the additional array of non-uniform features is separated from the second subset of the additional array of non-uniform features by a distance of at least about 2000 nanometers (nm). In some embodiments, the first subset of the additional array of non-uniform features is separated from the second subset of the additional array of non-uniform features by a distance of less than about 1000 nanometers (nm). In some embodiments, the first subset of the additional array of non-uniform features is parallel to the additional subset of the additional array of non-uniform features. In some embodiments, the first subset of the additional array of non-uniform features is separated from the second subset of the additional array of non-uniform features by one or more dielectric fins. In some embodiments, the first subset of the additional array of non-uniform features is not parallel to the second subset of the additional array of non-uniform features. In some embodiments, two or more non-uniform features of the additional array comprise a nanogap. In some embodiments, three or more non-uniform features of the additional array comprise a nanogap. In some embodiments, each feature of the additional array comprises a nanogap. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the non-uniform features of the additional array comprise one or more materials from the group consisting of silicon, silicon nitride, aluminum nitride, titanium dioxide, silicon dioxide, gallium nitride, hafnium oxide, germanium, and silicon carbide. In some embodiments, the chip is provided on a substrate. In some embodiments, the substrate comprises one or more materials from the group consisting of germanium, aluminum oxide, silicon dioxide, fused silica, silicon dioxide on silicon, silicon, silicon nitride, gallium nitride, calcium fluoride, and beryllium oxide. In some embodiments, two or more of the non-uniform features of the additional array are parallel to one another. In some embodiments, two or more of the non-uniform features of the additional array are nonparallel to one another. In some embodiments, the non-uniform features of the additional array are rectangular. In some embodiments, the non-uniform features of the additional array are rounded. In some embodiments, the non-uniform features of the additional array are arranged in a periodic configuration. In some embodiments, the non-uniform features of the additional array are arranged in a nonperiodic configuration. In some embodiments, a non-uniform feature of the additional array is a photonic crystal mirror.

In another aspect, the present disclosure provides a method of filtering a sample comprising: (a) providing a biological sample comprising one or more components on a chip, the chip comprising an array of non-uniform features configured to filter the one or more components according to size, and wherein the array of non-uniform features are interspersed with a plurality of electrodes or functionalized features configured to filter the one or more components according to charge or size; and (b) using the chip to filter the sample comprising one or more components.

In some embodiments, a feature of the array of non-uniform features comprises an electrical insulator or a semiconductor. In some embodiments, a feature of the array of non-uniform features comprises a metal. In some embodiments, the biological sample is a tissue sample. In some embodiments, the biological sample is a single cell. In some embodiments, the biological sample comprises a polynucleotide. In some embodiments, the biological sample comprises a polypeptide. In some embodiments, the biological sample comprises a metabolite. In some embodiments, the biological sample is an organism. In some embodiments, the organism is a bacterium. In some embodiments, the organism is a virus. In some embodiments, the biological sample comprises a cell fragment. In some embodiments, the method comprises an additional array of non-uniform features, wherein at least one feature of the additional array comprises a nanogap. In some embodiments, the nanogap is configured to concentrate an incident light. In some embodiments, the method comprises bringing the biological sample in contact with the nanogap. In some embodiments, the nanogap comprises a binding moiety that is specific or non-specific for the analyte. In some embodiments, the nanogap comprises a binding moiety with binding specificity for the sample. In some embodiments, the method comprises exposing the chip to a first light from a light source, such that the first light interacts with the array of non-uniform features and is further concentrated in the nanogap. In some embodiments, the first light is a laser. In some embodiments, the first light is a light emitting diode (LED) light. In some embodiments, the first light is a lamp. In some embodiments, the method comprises detecting a second light from the array of non-uniform features subsequent to the array of non-uniform features being exposed to the first light. In some embodiments, the method comprises using the second light to detect or identify an analyte in the biological sample. In some embodiments, the incident laser is at a wavelength of at least about 400 nanometers (nm) to at least about 1500 nanometers (nm). In some embodiments, the second light yields a vibrational scattering signature associated with the sample on the chip. In some embodiments, the vibrational scattering signature is a Raman spectrum. In some embodiments, the method comprises detecting the presence or absence of an analyte in the sample. In some embodiments, at least one non-uniform feature of the additional array has a height of at least about 50 nanometers (nm) to at least about 1000 nanometers (nm). In some embodiments, at least one non-uniform feature of the additional array has a width of at least about 50 nanometers (nm) to at least about 500 nanometers (nm). In some embodiments, at least one non-uniform feature of the additional array has a length of at least about 50 nanometers (nm) to at least about 2000 nanometers (nm). In some embodiments, the distance between the non-uniform features of the additional array is about 50 nanometers (nm) to about 1000 nanometers (nm). In some embodiments, the method comprises a first subset of the additional array of non-uniform features and a second subset of the additional array of non-uniform features, wherein the first subset and the second subset are adjacent to one another. In some embodiments, the first subset of the additional array of non-uniform features is separated from the second subset of the additional array of non-uniform features by a distance of at least about 5 nanometers (nm) to at most about 3000 nanometers (nm). In some embodiments, the first subset of the additional array of non-uniform features is separated from the second subset of the additional array of non-uniform features by a distance of at least about 2000 nanometers (nm). In some embodiments, the first subset of the additional array of non-uniform features is separated from the second subset of the additional array of non-uniform features by a distance of less than about 1000 nanometers (nm). In some embodiments, the first subset of the additional array of non-uniform features is parallel to the second subset of the additional array of non-uniform features. In some embodiments, the first subset of the additional array of non-uniform features is separated from the second subset of the additional array of non-uniform features by one or more dielectric fins. In some embodiments, the first subset of the additional array of non-uniform features is not parallel to the second subset of the additional array of non-uniform features. In some embodiments, two or more non-uniform features of the additional array comprise a nanogap. In some embodiments, three or more non-uniform features of the additional array comprise a nanogap. In some embodiments, each feature of the additional array comprises a nanogap. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the non-uniform features of the additional array comprise one or more materials from the group consisting of silicon, silicon nitride, aluminum nitride, titanium dioxide, silicon dioxide, gallium nitride, hafnium oxide, germanium, and silicon carbide. In some embodiments, the chip is provided on a substrate. In some embodiments, the substrate comprises one or more materials from the group consisting of aluminum oxide, silicon dioxide, fused silica, silicon dioxide on silicon, silicon, silicon nitride, gallium nitride, calcium fluoride, and beryllium oxide. In some embodiments, two or more of the non-uniform features of the additional array are parallel to one another. In some embodiments, two or more of the non-uniform features of the additional array are nonparallel to one another. In some embodiments, the non-uniform features of the additional array are rectangular. In some embodiments, the non-uniform features of the additional array are rounded. In some embodiments, the non-uniform features of the additional array are arranged in a periodic configuration. In some embodiments, the non-uniform features of the additional array are arranged in a nonperiodic configuration. In some embodiments, a non-uniform feature of the additional array is a photonic crystal mirror. In some embodiments, the method comprises providing a detector. In some embodiments, the method comprises using the detector to scan wavelengths in the detector plane. In some embodiments, the method comprises super resolution imaging. In some embodiments, the super resolution imaging is structured illumination microscopy (SIM). In some embodiments, the super resolution imaging is entropy based super resolution imaging (ESI). In some embodiments, the super resolution imaging is stochastic optical reconstruction microscopy (STORM). In some embodiments, the super resolution imaging is super resolution optical fluctuation imaging (SOFI). In some embodiments, the super resolution imaging is stimulated emission depletion microscopy (STED). In some embodiments, the method comprises producing one or more hyperspectral images. In some embodiments, each of the one or more hyperspectral images represents a distinct Raman signature. In some embodiments, the method comprises collecting data from the one or more hyperspectral images. In some embodiments, the method comprises developing a machine learning model. In some embodiments, a computing system is configured to execute the machine learning model. In some embodiments, the computing system is a neural network. In some embodiments, the method comprises developing a convolutional neural network (CNN).

In another aspect, the present disclosure provides a method of detecting or identifying an analyte's interactions with a sample, the method comprising: (a) providing the analyte on a chip comprising an array of non-uniform features, wherein a feature of the array of non-uniform features comprises an electrical insulator or a semiconductor, wherein the feature comprises a nanogap; (b) introducing a sample on the chip; (c) exposing the chip to a first light from a light source, such that the first light interacts with the array of non-uniform features and is further concentrated in the nanogap; (d) detecting a second light from the array of non-uniform features subsequent to the array of non-uniform features being exposed to the first light; and (e) collecting a time series of the second light.

In some embodiments, the method comprises following the real-time interactions of the analyte with the sample. In some embodiments, the sample comprises a protein. In some embodiments, the sample comprises a small molecule, peptide, or lipid. In some embodiments, a feature of the array of non-uniform features comprises an electrical insulator or a semiconductor. In some embodiments, a feature of the array of non-uniform features comprises a metal. In some embodiments, the analyte is a protein, polypeptide, or peptide. In some embodiments, the nanogap comprises a binding moiety that is non-specific for the analyte. In some embodiments, the nanogap comprises a binding moiety with binding specificity for the sample. In some embodiments, the first light is a laser. In some embodiments, the first light is a light emitting diode (LED) light. In some embodiments, the first light is a lamp. In some embodiments, the laser is at a wavelength of at least about 400 nanometers (nm) to at least about 1500 nanometers (nm). In some embodiments, the second light yields a vibrational scattering signature associated with the sample on the chip. In some embodiments, the vibrational scattering signature is a Raman spectrum. In some embodiments, at least one non-uniform feature of the additional array has a height of at least about 50 nanometers (nm) to at least about 1000 nanometers (nm). In some embodiments, at least one non-uniform feature of the additional array has a width of at least about 50 nanometers (nm) to at least about 500 nanometers (nm). In some embodiments, at least one non-uniform feature of the additional array has a length of at least about 50 nanometers (nm) to at least about 2000 nanometers (nm). In some embodiments, the distance between the non-uniform features of the additional array is about 50 nanometers (nm) to about 1000 nanometers (nm). In some embodiments, the method comprises a first subset of the additional array of non-uniform features and a second subset of the additional array of non-uniform features, wherein the first subset and the second subset are adjacent to one another. In some embodiments, the first subset of the additional array of non-uniform features is separated from the second subset of the additional array of non-uniform features by a distance of at least about 5 nanometers (nm) to at most about 3000 nanometers (nm). In some embodiments, the first subset of the additional array of non-uniform features is separated from the second subset of the additional array of non-uniform features by a distance of at least about 2000 nanometers (nm). In some embodiments, the first subset of the additional array of non-uniform features is separated from the second subset of the additional array of non-uniform features by a distance of less than about 1000 nanometers (nm). In some embodiments, the first subset of the additional array of non-uniform features is parallel to the second subset of the additional array of non-uniform features. In some embodiments, the first subset of the additional array of non-uniform features is separated from the second subset of the additional array of non-uniform features by one or more dielectric fins. In some embodiments, the first subset of the additional array of non-uniform features is not parallel to the second subset of the additional array of non-uniform features. In some embodiments, two or more non-uniform features of the additional array comprise a nanogap. In some embodiments, three or more non-uniform features of the additional array comprise a nanogap. In some embodiments, each feature of the additional array comprises a nanogap. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the non-uniform features of the additional array comprise one or more materials from the group consisting of silicon, silicon nitride, aluminum nitride, titanium dioxide, silicon dioxide, gallium nitride, hafnium oxide, germanium, and silicon carbide. In some embodiments, the chip is provided on a substrate. In some embodiments, the substrate comprises one or more materials from the group consisting of aluminum oxide, silicon dioxide, fused silica, silicon dioxide on silicon, silicon, silicon nitride, gallium nitride, calcium fluoride, and beryllium oxide. In some embodiments, two or more of the non-uniform features of the additional array are parallel to one another. In some embodiments, two or more of the non-uniform features of the additional array are nonparallel to one another. In some embodiments, the non-uniform features of the additional array are rectangular. In some embodiments, the non-uniform features of the additional array are rounded. In some embodiments, the non-uniform features of the additional array are arranged in a periodic configuration. In some embodiments, the non-uniform features of the additional array are arranged in a nonperiodic configuration. In some embodiments, a non-uniform feature of the additional array is a photonic crystal mirror. In some embodiments, the method comprises providing a detector. In some embodiments, the method comprises using the detector to scan wavelengths in the detector plane. In some embodiments, the method comprises super resolution imaging. In some embodiments, the super resolution imaging is structured illumination microscopy (SIM). In some embodiments, the super resolution imaging is entropy based super resolution imaging (ESI). In some embodiments, the super resolution imaging is stochastic optical reconstruction microscopy (STORM). In some embodiments, the super resolution imaging is super resolution optical fluctuation imaging (SOFI). In some embodiments, the super resolution imaging is stimulated emission depletion microscopy (STED). In some embodiments, the method comprises producing one or more hyperspectral images. In some embodiments, each of the one or more hyperspectral images represents a distinct Raman signature. In some embodiments, the method comprises collecting data from the one or more hyperspectral images. In some embodiments, the method comprises developing a machine learning model. In some embodiments, a computer system is configured to execute the machine learning model. In some embodiments, the computing system is a neural network. In some embodiments, the method comprises developing a convolutional neural network (CNN). A chip for analyzing a biological or chemical sample, the chip comprising two or more resonators, wherein each of the two or more resonators supports one or more guided modes, wherein each of the two or more resonators has a corresponding longitudinal perturbation, wherein an incident light is coupled to two or more of the guided mode resonances by the longitudinal perturbations of the resonators, wherein each resonator comprises an electrical insulator or a semiconductor, wherein each resonator comprises at least one nanogap configured to concentrate an incident light, wherein one or more regions of high electromagnetic field intensity are localized within and in proximity to each nanogap, whereby environmental sensing is provided. In some embodiments, the chip comprises an optical source configured to provide free-space or totally-internally-reflected radiation, wherein the incident light is an incident laser. In some embodiments, the incident laser is at a wavelength of at least about 400 nanometers (nm) to at least about 1800 nanometers (nm). In some embodiments, the incident light is a light emitting diode (LED) light. In some embodiments, the incident light is a lamp. In some embodiments, the resonator comprises a binding moiety that is non-specific for the analyte. In some embodiments, the resonator comprises a binding moiety with binding specificity for the analyte. In some embodiments, the resonator contains no binding moiety In some embodiments, the biological or chemical sample comprises two or more components. In some embodiments, the sample is a tissue section. In some embodiments, the sample is a liquid or dissolved solid. In some embodiments, the analyte is a single cell or collection of cells. In some embodiments, the cells are prokaryotic or eukaryotic In some embodiments, the analyte is a virus. In some embodiments, the analyte is an oligonucleotide or polynucleotide. In some embodiments, the analyte is a polypeptide, or protein. In some embodiments, the analyte is a small molecule, such as a nucleic acid, amino acid, or metabolite. In some embodiments, the analyte is a polymer or microplastic. In some embodiments, at least one feature of the resonator has a height of at least about 50 nanometers (nm) to at least about 1000 nanometers (nm). In some embodiments, at least one feature of the resonator has a width of at least about 50 nanometers (nm) to at least about 2000 nanometers (nm). In some embodiments, at least one feature of the resonator has a length of at least about 50 nanometers (nm) to at least about 2000 nanometers (nm). In some embodiments, the distance between the resonators is about 50 nanometers (nm) to about 1,000 nanometers (nm). In some embodiments, the first subset of the array of non-uniform features is separated from the second subset of the array of non-uniform features by a distance of at least about 5 nanometers (nm) to at most about 20,000 nanometers (nm). In some embodiments, resonators are separated from each other by one or more dielectric fins. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the resonators comprise one or more materials from the group consisting of silicon, silicon nitride, aluminum nitride, titanium dioxide, silicon dioxide, gallium nitride, hafnium oxide, germanium, and silicon carbide. In some embodiments, the resonators are on a substrate. In some embodiments, the substrate comprises one or more materials from the group consisting of germanium aluminum oxide, silicon dioxide, fused silica, silicon dioxide on silicon, silicon, silicon nitride, gallium nitride, calcium fluoride, and beryllium oxide. In some embodiments, two or more resonators of the array are parallel to one another. In some embodiments, two or more resonators of the array are nonparallel to one another. In some embodiments, the non-uniform features of the array are arranged in a periodic configuration. In some embodiments, the non-uniform features of the array are arranged in a nonperiodic configuration. In some embodiments, each resonator further contains a photonic crystal mirror designed to confine the guided mode resonance.

In another aspect, the present disclosure provides a method of detecting or identifying an analyte in a biological or chemical sample, comprising: (a) providing the biological or chemical sample on a chip comprising two or more resonators, wherein each of the two or more resonators supports one or more guided modes; wherein each of the two or more resonators has a corresponding longitudinal perturbation, where at least one guided mode resonance is supported in each resonator; wherein an incident light is coupled to two or more of the guided mode resonances by the longitudinal perturbations of the resonators; a feature of the array of non-uniform features wherein each resonator comprises an electrical insulator or a semiconductor; wherein each resonator comprises at least one, wherein the feature comprises a nanogap configured to concentrate an incident light; wherein one or more regions of high electromagnetic field intensity are localized within and in proximity to each nanogap, whereby environmental sensing is provided; (b) exposing the chip to a first light from a light source, such that the first light interacts with resonators and is further concentrated in the nanogaps; (c) detecting a second light from resonators subsequent to the array of non-uniform features being exposed to the first light; and (d) using the second light to detect or identify the analyte.

In some embodiments, the second light yields a vibrational scattering signature associated with the analyte. In some embodiments, the vibrational scattering signature is a Raman spectrum. In some embodiments, the vibrational scattering signature is an Infrared spectrum In some embodiments, the second light is autofluorescence associated with the analyte. In some embodiments, the light source of the first light is integrated with the chip. In some embodiments, the light source of the first light is not integrated with the chip. In some embodiments, the second light is detected with an integrated spectrometer or filter system on the chip. In some embodiments, the light source of the second light is detected with spectrometers or filters not integrated with the chip. In some embodiments, the method comprises detecting the presence or absence of the analyte. In some embodiments, the resonator comprises a binding moiety that is non-specific for the analyte. In some embodiments, the resonator comprises a binding moiety with binding specificity for the analyte. In some embodiments, the resonator comprises no binding moiety. In some embodiments, the incident light is an incident laser. In some embodiments, the incident laser is at a wavelength of at least about 400 nanometers (nm) to at least about 1800 nanometers (nm). In some embodiments, the incident light is a light emitting diode (LED) light. In some embodiments, the incident light is a lamp. In some embodiments, the method comprises using the detector to scan wavelengths in the detector plans. In some embodiments, the method comprises using super resolution imaging to image at least a portion of the chip. In some embodiments, the super resolution imaging is structured illumination microscopy (SIM). In some embodiments, the super resolution imaging is entropy based super resolution imaging (ESI). In some embodiments, the super resolution imaging is stochastic optical reconstruction microscopy (STORM). In some embodiments, the super resolution imaging is super resolution optical fluctuation imaging (SOFI). In some embodiments, the super resolution imaging is stimulated emission depletion microscopy (STED). In some embodiments, the method comprises producing one or more hyperspectral images. In some embodiments, each of the one or more hyperspectral images represents a distinct Raman signature. In some embodiments, the method comprises collecting data from the one or more hyperspectral images. In some embodiments, the method comprises developing a machine learning model. In some embodiments, the computing system is a neural network. In some embodiments, the neural network is a convolutional neural network (CNN). In some embodiments, the biological or chemical sample comprises two or more components. In some embodiments, the biological sample is a tissue sample. In some embodiments, the sample is a liquid or dissolved solid. In some embodiments, the analyte is a single cell or collection of cells. In some embodiments, the cells are prokaryotic or eukaryotic In some embodiments, the analyte is a virus. In some embodiments, the analyte is an oligonucleotide or polynucleotide. In some embodiments, the analyte is a polypeptide, or protein In some embodiments, the analyte is a small molecule, such as a nucleic acid, amino acid, or metabolite. In some embodiments, the analyte is a polymer or microplastic In some embodiments, at least one feature of the resonator has a height of at least about 50 nanometers (nm) to at least about 1000 nanometers (nm). In some embodiments, at least one feature of the resonator has a width of at least about 50 nanometers (nm) to at least about 2000 nanometers (nm). In some embodiments, at least one feature of the resonator has a length of at least about 50 nanometers (nm) to at least about 2000 nanometers (nm). In some embodiments, the distance between the resonators is about 50 nanometers (nm) to about 1,000 nanometers (nm). In some embodiments, the first subset of the array of non-uniform features is separated from the second subset of the array of non-uniform features by a distance of at least about 5 nanometers (nm) to at most about 20,000 nanometers (nm). In some embodiments, resonators are separated from each other by one or more dielectric fins. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the resonators comprise one or more materials from the group consisting of silicon, silicon nitride, aluminum nitride, titanium dioxide, silicon dioxide, gallium nitride, hafnium oxide, germanium, and silicon carbide. In some embodiments, the resonators are on a substrate. In some embodiments, the substrate comprises one or more materials from the group consisting of germanium aluminum oxide, silicon dioxide, fused silica, silicon dioxide on silicon, silicon, silicon nitride, gallium nitride, calcium fluoride, and beryllium oxide. In some embodiments, two or more resonators of the array are parallel to one another. In some embodiments, two or more resonators of the array are nonparallel to one another. In some embodiments, the non-uniform features of the array are arranged in a periodic configuration. In some embodiments, the non-uniform features of the array are arranged in a nonperiodic configuration. In some embodiments, each resonator further contains a photonic crystal mirror designed to confine the guided mode resonance.

A chip-based method of filtering a sample prior to detecting or identifying an analyte, comprising: (a) providing a biological or chemical sample comprising one or more components on a chip, the chip comprising an array of non-uniform features configured to filter the one or more components according to size, and wherein the array of non-uniform features are interspersed with a plurality of electrodes or functionalized features configured to filter the one or more components according to charge, or size, or chemical/biological affinity; and (b) using the chip to filter the sample comprising one or more components; (c) providing the filtered sample on a sensor region on the same chip, the sensor region comprising two or more resonators, wherein each of the two or more resonators supports one or more guided modes; wherein each of the two or more resonators has a corresponding longitudinal perturbation, where at least one guided mode resonance is supported in each resonator; wherein an incident light is coupled to two or more of the guided mode resonances by the longitudinal perturbations of the resonators; a feature of the array of non-uniform features wherein each resonator comprises an electrical insulator or a semiconductor; wherein each resonator comprises at least one, wherein the feature comprises a nanogap configured to concentrate an incident light; wherein one or more regions of high electromagnetic field intensity are localized within and in proximity to each nanogap, whereby environmental sensing is provided; (d) exposing the sensor region to a first light from a light source, such that the first light interacts with resonators and is further concentrated in the nanogaps; (e) detecting a second light from resonators subsequent to the array of non-uniform features being exposed to the first light; and (f) using the second light to detect or identify the analyte.

In some embodiments, a feature of the array of non-uniform features comprises an electrical insulator or a semiconductor. In some embodiments, a feature of the array of non-uniform features comprises a metal. In some embodiments, the sample is a liquid or dissolved solid. In some embodiments, analytes in the sample are cells. In some embodiments, the cells are prokaryotic or eukaryotic In some embodiments, analytes in the sample are viruses. In some embodiments, analytes in the sample are oligonucleotides or polynucleotides. In some embodiments, analytes in the sample are polypeptides, or proteins. In some embodiments, analytes in the sample are small molecules, such as nucleic acids, amino acids, or metabolites. In some embodiments, analytes in the sample are polymer or microplastic. In some embodiments, the array is composed of dielectric or semiconducting features with dimensions of 50 to 20000 nm. In some embodiments, the array features are arranged with distances of 50 nm to 20000 nm. In some embodiments, the features are arranged in a two dimensional or three-dimensional array. In some embodiments, the features are interspersed with metal electrodes to filter the sample by charge. In some embodiments, the features comprise one or more materials from the group consisting of silicon, silicon nitride, aluminum nitride, titanium dioxide, silicon dioxide, gallium nitride, hafnium oxide, germanium, and silicon carbide. In some embodiments, the chip is provided on a substrate. In some embodiments, the substrate comprises one or more materials from the group consisting of aluminum oxide, silicon dioxide, fused silica, silicon dioxide on silicon, silicon, silicon nitride, gallium nitride, calcium fluoride, and beryllium oxide. In some embodiments, the features are chemically functionalized with binding moieties to filter the sample by chemical or biological affinity. In some embodiments, the filtered sample is introduced to the sensor region for analysis and detection. In some embodiments, the second light yields a vibrational scattering signature associated with the analyte. In some embodiments, the vibrational scattering signature is a Raman spectrum. In some embodiments, the vibrational scattering signature is an Infrared spectrum. In some embodiments, the second light is autofluorescence associated with the analyte. In some embodiments, the light source of the first light is integrated with the chip. In some embodiments, the light source of the first light is not integrated with the chip. In some embodiments, the second light is detected with an integrated spectrometer or filter system on the chip. In some embodiments, the light source of the second light is detected with spectrometers or filters not integrated with the chip. In some embodiments, the method further comprises detecting the presence or absence of the analyte. In some embodiments, the resonator comprises a binding moiety that is non-specific for the analyte. In some embodiments, the resonator comprises a binding moiety with binding specificity for the analyte. In some embodiments, the incident light is an incident laser. In some embodiments, the incident laser is at a wavelength of at least about 400 nanometers (nm) to at least about 1800 nanometers (nm). In some embodiments, the incident light is a light emitting diode (LED) light. In some embodiments, the incident light is a lamp. In some embodiments, the method comprises using the detector to scan wavelengths in the detector plans. In some embodiments, the method comprises using super resolution imaging to image at least a portion of the chip. In some embodiments, the super resolution imaging is structured illumination microscopy (SIM). In some embodiments, the super resolution imaging is entropy based super resolution imaging (ESI). In some embodiments, the super resolution imaging is stochastic optical reconstruction microscopy (STORM). In some embodiments, the super resolution imaging is super resolution optical fluctuation imaging (SOFI). In some embodiments, the super resolution imaging is stimulated emission depletion microscopy (STED). In some embodiments, the method comprises producing one or more hyperspectral images. In some embodiments, each of the one or more hyperspectral images represents a distinct Raman signature. In some embodiments, the method comprises collecting data from the one or more hyperspectral images. In some embodiments, the method comprises developing a machine learning model. In some embodiments, a computing system is configured to execute the machine learning model. In some embodiments, the computing system is a neural network. In some embodiments, the neural network is a convolutional neural network (CNN). In some embodiments, at least one feature of the resonator has a height of at least about 50 nanometers (nm) to at least about 1000 nanometers (nm). In some embodiments, at least one feature of the resonator has a width of at least about 50 nanometers (nm) to at least about 2000 nanometers (nm). In some embodiments, at least one feature of the resonator has a length of at least about 50 nanometers (nm) to at least about 2000 nanometers (nm). In some embodiments, the distance between the resonators is about 50 nanometers (nm) to about 1,000 nanometers (nm). In some embodiments, the first subset of the array of non-uniform features is separated from the second subset of the array of non-uniform features by a distance of at least about 5 nanometers (nm) to at most about 20,000 nanometers (nm). In some embodiments, resonators are separated from each other by one or more dielectric fins. In some embodiments, the nanogap is at least about 5 nanometers (nm) to at most about 150 nm wide. In some embodiments, the nanogap is at least about 5 nm to at most about 70 nm wide. In some embodiments, the nanogap is at least about 5 nm to at most about 30 nm wide. In some embodiments, the resonators comprise one or more materials from the group consisting of silicon, silicon nitride, aluminum nitride, titanium dioxide, silicon dioxide, gallium nitride, hafnium oxide, germanium, and silicon carbide. In some embodiments, the resonators are on a substrate. In some embodiments, the substrate comprises one or more materials from the group consisting of germanium aluminum oxide, silicon dioxide, fused silica, silicon dioxide on silicon, silicon, silicon nitride, gallium nitride, calcium fluoride, and beryllium oxide. In some embodiments, two or more resonators of the array are parallel to one another. In some embodiments, two or more resonators of the array are nonparallel to one another. In some embodiments, the non-uniform features of the array are arranged in a periodic configuration. In some embodiments, the non-uniform features of the array are arranged in a nonperiodic configuration. In some embodiments, each resonator further contains a photonic crystal mirror designed to confine the guided mode resonance.

In another aspect, the present disclosure provides a method of detecting or identifying an analyte's interactions with a sample, the method comprising: (a) providing the analyte on a chip comprising two or more resonators, wherein each of the two or more resonators supports one or more guided modes; wherein each of the two or more resonators has a corresponding longitudinal perturbation, where at least one guided mode resonance is supported in each resonator; wherein an incident light is coupled to two or more of the guided mode resonances by the longitudinal perturbations of the resonators; a feature of the array of non-uniform features wherein each resonator comprises an electrical insulator or a semiconductor; wherein each resonator comprises at least one, wherein the feature comprises a nanogap configured to concentrate an incident light; wherein one or more regions of high electromagnetic field intensity are localized within and in proximity to each nanogap, whereby environmental sensing is provided; (b) introducing a sample on the chip; (c) exposing the chip to a first light from a light source, such that the first light interacts with the array of resonators; (d) detecting a second light from resonators subsequent to the resonators being exposed to the first light; and (e) collecting a time series of the second light.

In some embodiments, the method comprises following the real-time interactions of the analyte with the sample. In some embodiments, the sample comprises a macromolecule, such as a protein or biologic. In some embodiments, the sample comprises a molecule. In some embodiments, the analyte is a protein, antibody, gene fragment, virus, cell, microplastic, or polymer. In some embodiments, the nanogap comprises a binding moiety that is non-specific for the analyte. In some embodiments, the nanogap comprises a binding moiety with binding specificity for the sample. In some embodiments, the first light is a laser. In some embodiments, the first light is a light emitting diode (LED) light. In some embodiments, the laser is at a wavelength of at least about 400 nanometers (nm) to at least about 1800 nanometers (nm). In some embodiments, the second light yields a vibrational scattering signature associated with the sample on the chip. In some embodiments, the vibrational scattering signature is a Raman spectrum. In some embodiments, the vibrational scattering signature is an Infrared spectrum In some embodiments, the second light is autofluorescence. In some embodiments, at least one feature of the resonator has a height of at least about 50 nanometers (nm) to at least about 1000 nanometers (nm). In some embodiments, at least one feature of the resonator has a width of at least about 50 nanometers (nm) to at least about 2000 nanometers (nm). In some embodiments, at least one feature of the resonator has a length of at least about 50 nanometers (nm) to at least about 2000 nanometers (nm). In some embodiments, the distance between the resonators is about 50 nanometers (nm) to about 1,000 nanometers (nm). In some embodiments, the first subset of the array of non-uniform features is separated from the second subset of the array of non-uniform features by a distance of at least about 5 nanometers (nm) to at most about 20,000 nanometers (nm). In some embodiments, resonators are separated from each other by one or more dielectric fins. In some embodiments, the nanogap is at least about 5 nanometers (nm) to at most about 150 nm wide. In some embodiments, the resonators comprise one or more materials from the group consisting of silicon, silicon nitride, aluminum nitride, titanium dioxide, silicon dioxide, gallium nitride, hafnium oxide, germanium, and silicon carbide. In some embodiments, the resonators are on a substrate. In some embodiments, the substrate comprises one or more materials from the group consisting of germanium aluminum oxide, silicon dioxide, fused silica, silicon dioxide on silicon, silicon, silicon nitride, gallium nitride, calcium fluoride, and beryllium oxide. In some embodiments, two or more resonators of the array are parallel to one another. In some embodiments, two or more resonators of the array are nonparallel to one another. In some embodiments, the non-uniform features of the array are arranged in a periodic configuration. In some embodiments, the non-uniform features of the array are arranged in a nonperiodic configuration. In some embodiments, each resonator further contains a photonic crystal mirror designed to confine the guided mode resonance. In some embodiments, the method comprises providing a detector. In some embodiments, the method comprises using the detector to scan wavelengths in the detector plane. In some embodiments, the method comprises super resolution imaging. In some embodiments, the super resolution imaging is structured illumination microscopy (SIM). In some embodiments, the super resolution imaging is entropy based super resolution imaging (ESI). In some embodiments, the super resolution imaging is stochastic optical reconstruction microscopy (STORM). In some embodiments, the super resolution imaging is super resolution optical fluctuation imaging (SOFI). In some embodiments, the super resolution imaging is stimulated emission depletion microscopy (STED). In some embodiments, the method comprises producing one or more hyperspectral images. In some embodiments, each of the one or more hyperspectral images represents a distinct analyte-sample interaction state. In some embodiments, the method comprises collecting time-dependent data from the one or more hyperspectral images.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 depicts an example of a processing workflow, according to some embodiments.

FIG. 2A depicts exemplary designs of an array of non-uniform features, according to some embodiments.

FIG. 2B depicts an example view of an array design, according to some embodiments.

FIGS. 3A-3F depict alternative designs for arrays of non-uniform features, according to some embodiments.

FIG. 4 depicts an example of a two-dimensional array of non-uniform features, according to some embodiments.

FIGS. 5A-5B depict examples of two-dimensional arrays, according to some embodiments.

FIG. 6A depicts the portions of an array, according to some embodiments.

FIG. 6B depict an example of the field enhancement of an array not comprising a nanogap, according to some embodiments.

FIGS. 7A-7B depict an example of a field enhancement calculation and an inset zoom of an array comprising a plurality of gaps, according to some embodiments.

FIG. 8A depicts an example of an array with a single nanogap, according to some embodiments.

FIG. 8B depicts an example field profile for the array of FIG. 8A, according to some embodiments.

FIG. 9 depicts an example of a chip comprising a detection region and a separation region, according to some embodiments.

FIGS. 10A-10C depict a pathway for analysis of the spectra of the present disclosure, according to some embodiments.

FIG. 11 depicts an example of tissue mapping with an array, according to some embodiments.

FIG. 12 depicts an example micrograph of a plurality of arrays, according to some embodiments.

FIG. 13 depicts a micrograph of an example array, according to some embodiments.

FIGS. 14A-14B depict micrographs of example pluralities of array, each array comprising a plurality of gaps, according to some embodiments.

FIG. 15 depicts an example of spectral measurement of an interaction, according to some embodiments.

FIG. 16 depicts sample Raman spectra, according to some embodiments.

FIG. 17 depicts a computer system that is programmed or otherwise configured to implement methods provided herein.

FIGS. 18A-18D show additional examples of fabricated arrays, according to some embodiments. FIGS. 18A and 18B depict example fabricated structures with gaps along full resonator. FIGS. 18C and 18D depict example fabricated structures with a single gap in the resonator.

FIGS. 19A-19B show examples of Raman spectra of proteins and protein fragments according to some embodiments.

FIGS. 20A-20C show examples of post translational modification spectra and associated confusion matrix, according to some embodiments.

FIG. 21 shows an example of a Raman emission versus excitation wavelength plot, according to some embodiments.

FIGS. 22A-22C show examples of fabricated arrays at different magnification levels, according to some embodiments.

FIGS. 23A-23C show far field scattering profiles of arrays, according to some embodiments.

FIGS. 24A-24B show examples of simulated clustered analysis of analytes and the associated simulated Raman spectra, according to some embodiments.

FIGS. 25A-25B show an example of a cluster analysis of a plurality of Raman spectra to identify analytes, according to some embodiments.

FIGS. 26A-26B show an example of Raman spectra and a difference spectrum associated with the introduction of a small molecule to a peptide, according to some embodiments.

FIGS. 27A-27D show an example of a confusion matrix and associated Raman spectra, according to some embodiments.

FIGS. 28A-28B show an example of a confusion matrix and associated Raman spectra, according to some embodiments.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Raman spectroscopy, including spontaneous Raman spectroscopy, stimulated Raman spectroscopy (SRS), and coherent anti-stokes Raman spectroscopy (CARS) provides numerous benefits such as, for example, strong sensitivity to the composition of an analyte. Increased light intensity can provide higher signal, which can improve the ability to discern differences in spectra and detect features of analytes. Arrays of dielectric features with a nanogap in the features can significantly increase light field, thereby providing high signal.

### Systems for Raman Spectroscopy

In certain aspects, described herein are systems and methods for processing a biological sample. The biological sample may comprise one or more components. In some embodiments, described herein is a chip for processing a biological sample. In some embodiments, the chip comprises an array of non-uniform features, wherein a feature of the array of non-uniform features comprises an electrical insulator or a semi-conductor. In some embodiments, the feature comprises a nanogap. In some embodiments, the array of non-uniform features is interspersed with a plurality of electrodes or functionalized features configured to filter the one or more components according to size or charge. In some embodiments, the chip comprises or more resonators, wherein each of the two or more resonators supports one or more guided modes, wherein each of the two or more resonators has a corresponding longitudinal perturbation, wherein an incident light is coupled to two or more of the guided mode resonances by the longitudinal perturbations of the resonators, wherein each resonator comprises an electrical insulator or a semiconductor, wherein each resonator comprises at least one nanogap configured to concentrate an incident light, wherein one or more regions of high electromagnetic field intensity are localized within and in proximity to each nanogap, whereby environmental sensing is provided.

### (i) Chips

In certain aspects, described herein are chips comprising an array of non-uniform features (e.g., a resonator comprising the array of non-uniform features). The array may comprise two or more non-uniform features. The features of the array may be parallel to one another. The features of the array may be nonparallel to each other. At least one feature of the array may be rectangular. At least one feature of the array may be rounded. The non-uniform features of the array may be arranged in a periodic configuration. The non-uniform features of the array may be arranged in a nonperiodic configuration. At least one non-uniform feature of the array may be a photonic crystal mirror. The array can be a resonator as described elsewhere herein. For example, the terms array of non-uniform features and resonator can be used interchangeably.

The non-uniform features described herein may comprise a nanogap. The nanogap may be configured to concentrate a light field coupled into the array. The nanogap may be formed as a part of the feature (e.g., formed at a same time as the feature). The nanogap may be formed after the feature (e.g., by removal of material from the feature).

In some embodiments, at least one of the non-uniform features of the array comprise a nanogap. In some embodiments, two or more non-uniform features of the array comprise a nanogap. In some embodiments, three or more non-uniform features of the array comprise a nanogap. In some embodiments, four or more non-uniform features of the array comprise a nanogap. In some embodiments, five or more non-uniform features of the array comprise a nanogap. In some embodiments, each of the non-uniform features of the array comprise a nanogap. In some embodiments, each non-uniform feature comprises about 1, 2, 3, 4, 5, 6, 7, 8, 9, or 10 nanogaps.

In some embodiments, the nanogap is configured to concentrate an incident light. The incident light may be an incident laser, a light emitting diode (LED) light, a lamp, or a combination thereof. The incident light may be an LED. The incident light may be a lamp. In some embodiments, a light source of a first light is integrated with the chip. In some embodiments, a light source of a first light is not integrated with the chip. In some embodiments, a light source of a second light is integrated with the chip. In some embodiments, a light source of a second light is not integrated with the chip.

The incident light may be an incident laser. The incident laser may have a wavelength of at least at least about 100 nanometers (nm), at least about 200 nm, at least about 300 nm, at least about 400 nm, at least about 500 nm, at least about 600 nm, at least about 700 nm, at least about 800 nm, at least about 900 nm, at least about 1000 nm, at least about 1100 nm, at least about 1200 nm, at least about 1300 nm, at least about 1400 nm, at least about 1500 nm, at least about 1600 nm, at least about 1700 nm, at least about 1800 nm, at least about 1900 nm, at least about or 2000 nm. The incident laser may have a wavelength of no more than about 100 nm, about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, about or 2000 nm. The incident laser may have a wavelength of at least about 400 nm to at least about 1800 nm. The incident laser may have a wavelength of at least about 100 nm to at least about 1000 nm. The incident laser may have a wavelength of at least about 300 nm to about 1800 nm, about 400 nm to about 1800 nm, about 500 nm to about 1800 nm, about 600 nm to about 1800 nm, about 700 nm to about 1800 nm, about 800 nm to about 1800 nm, about 900 nm to about 1800 nm, about 1000 nm to about 1800 nm, about 1100 nm to about 1800 nm, about 1200 nm to about 1800 nm, about 1300 nm to about 1800 nm, about 1400 nm to about 1800 nm, about 1500 nm to about 1800 nm, about 1600 nm to about 1800 nm or about 1700 nm to about 1800 nm. In some cases, the incident light can be generated by a plurality of light sources (e.g., lasers). For example, the incident light can be a mixture of light from two lasers. In another example, the incident light can be light from a first laser and subsequently light from a second laser. The use of a plurality of light sources can enable use of pump-probe type excitation or detection schemes (e.g., stimulated Raman scattering, coherent anti-Stokes Raman, etc.).

The nanogap may comprise a binding moiety that is specific for an analyte. The nanogap may comprise a binding moiety that is non-specific for an analyte. In some embodiments, the nanogap comprises a binding moiety with binding specificity for said analyte. The analyte may comprise a protein. The analyte may comprise an enzyme. The analyte may comprise a kinase. The analyte may comprise a receptor. The analyte may comprise a tyrosine kinase. The analyte may comprise a Janus kinase 3. The analyte may comprise an epidermal growth factor receptor (EGFR).

The nanogap may be at least about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about or about 200 nm wide. The nanogap may be no more than about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about or about 200 nm wide. The nanogap may be at least about 5 nm to at least about 200 nm, about at least about 5 nm to at least about 150 nm, about at least about 5 nm to at least about 100 nm, about at least about 5 nm to at least about 50 nm. The nanogap may be at least about 5 nm to at least about 150 nm.

In some embodiments, the chip comprises an additional array comprising one or more non-uniform features configured to filter two or more components of the biological sample according to size, charge, or binding affinity. In some embodiments, the additional array is configured to filter the two or more components according to size. In some embodiments, the additional array is configured to filter the two or more components according to charge. In some embodiments, the additional array is configured to filter the two or more components according to binding affinity. In some embodiments, the non-uniform features of said additional array are interspersed with a plurality of electrodes or functionalized features configured to filter the two or more components according to charge or size. In some embodiments, the functionalized feature comprises a functionalized oxide surface. In some embodiments, the array of non-uniform features is interspersed with a plurality of electrodes or functionalized features configured to filter the two or more components according to size or charge. In some embodiments, the biological sample comprises at least about 2, 3, 4, 5, 6, 7, 8, 9, 10, 20, 30, 40, 50, 60, 70, 80, 90, 100, 150, 200, 250, 300, 350, 400, 450, 500, or more components.

In some embodiments, a feature of the array comprises an electrical insulator or a semiconductor. In some embodiments, the feature comprises one or more materials from the group consisting of silicon, silicon nitride, aluminum nitride, titanium dioxide, silicon dioxide, gallium nitride, hafnium oxide, germanium, and silicon carbide. In some embodiments, the chip is provided on a substrate. In some embodiments, the substrate comprises one or more materials from the group consisting of germanium aluminum oxide, silicon dioxide, fused silica, silicon dioxide on silicon, silicon, silicon nitride, gallium nitride, calcium fluoride, and beryllium oxide.

At least one feature of the array described herein may have a height of at least about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about or about 2000 nm. In some embodiments, the height is no more than about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about or 2000 nm. In some embodiments, the height is at least about 10 nm to at least about 1000 nm. In some embodiments, the height is at least about 20 nm to at least about 1000 nm. In some embodiments, the height is at least about 30 nm to at least about 1000 nm. In some embodiments, the height is at least about 40 nm to at least about 1000 nm. In some embodiments, the height is at least about 10 nm to at least about 1000 nm. In some embodiments, the height is at least about 50 nm to at least about 1000 nm. In some embodiments, the height is at least about 60 nm to at least about 1000 nm. In some embodiments, the height is at least about 70 nm to at least about 1000 nm. In some embodiments, the height is at least about 80 nm to at least about 1000 nm. In some embodiments, the height is at least about 90 nm to at least about 1000 nm. In some embodiments, the height is at least about 100 nm to at least about 1000 nm.

At least one feature of the array described herein may have a width of at least about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about or 2000 nm. In some embodiments, the width is no more than about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about or 2000 nm. In some embodiments, the width is at least about 10 nm to at least about 500 nm. In some embodiments, the width is at least about 50 nm to at least about 500 nm. In some embodiments, the width is at least about 50 nm to at least about 1000 nm.

At least one feature of the array described herein may have a width of at least about 10 nm, about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about or 2000 nm. In some embodiments, the width is no more than about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about or 2000 nm. In some embodiments, the length is at least about 50 nm to at least about 2000 nm. In some embodiments, the length is at least about 100 nm to at least about 2000 nm. In some embodiments, the length is at least about 200 nm to at least about 2000 nm. In some embodiments, the length is at least about 300 nm to at least about 2000 nm. In some embodiments, the length is at least about 400 nm to at least about 2000 nm. In some embodiments, the length is at least about 500 nm to at least about 2000 nm. In some embodiments, the length is at least about 600 nm to at least about 2000 nm. In some embodiments, the length is at least about 700 nm to at least about 2000 nm. In some embodiments, the length is at least about 800 nm to at least about 2000 nm. In some embodiments, the length is at least about 900 nm to at least about 2000 nm. In some embodiments, the length is at least about 1000 nm to at least about 2000 nm.

The non-uniform features described herein may be separated. In some embodiments, the distance between the non-uniform features is at least about 10 nm, at least about 20 nm, at least about 30 nm, at least about 40 nm, at least about 50 nm, at least about 60 nm, at least about 70 nm, at least about 80 nm, at least about 90 nm, at least about 100 nm, at least about 110 nm, at least about 120 nm, at least about 130 nm, at least about 140 nm, at least about 150 nm, at least about 160 nm, at least about 170 nm, at least about 180 nm, at least about 190 nm, at least about 200 nm, at least about 250 nm, at least about 300 nm, at least about 350 nm, at least about 400 nm, at least about 450 nm, at least about 500 nm, at least about 550 nm, at least about 600 nm, at least about 650 nm, at least about 700 nm, at least about 750 nm, at least about 800 nm, at least about 850 nm, at least about 900 nm, at least about 950 nm, at least about 1000 nm, at least about 1100 nm, at least about 1200 nm, at least about 1300 nm, at least about 1400 nm, at least about 1500 nm, at least about 1600 nm, at least about 1700 nm, at least about 1800 nm, at least about 1900 nm, or at least about 2000 nm. In some embodiments, the distance between the non-uniform features is no more than about 20 nm, about 30 nm, about 40 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about or 2000 nm. In some embodiments, the distance is at least about 10 nm to at least about 1000 nm. In some embodiments, the distance is at least about 20 nm to at least about 1000 nm. In some embodiments, the distance is at least about 30 nm to at least about 1000 nm. In some embodiments, the distance is at least about 40 nm to at least about 1000 nm. In some embodiments, the distance is at least about 10 nm to at least about 1000 nm. In some embodiments, the distance is at least about 50 nm to at least about 1000 nm. In some embodiments, the distance is at least about 60 nm to at least about 1000 nm. In some embodiments, the distance is at least about 70 nm to at least about 1000 nm. In some embodiments, the distance is at least about 80 nm to at least about 1000 nm. In some embodiments, the distance is at least about 90 nm to at least about 1000 nm. In some embodiments, the distance is at least about 100 nm to at least about 1000 nm.

Two or more of the non-uniform features of the array may be parallel to one another. Two or more of the non-uniform features of the array may be non-parallel to each other.

The chip may comprise a first subset of an array of non-uniform features and a second subset of an array of non-uniform features. The first subset and the second subset may be adjacent to each other. The first subset and the second subset may be parallel to each other. The first subset may be parallel to the second subset. In some embodiments, the first subset is not parallel to the second subset. In some embodiments, the first subset is separated from the second subset by one or more dielectric fins.

The first subset and the second subset may be separated by a distance of at least about 1 nm, about 2 nm, about 3 nm, about 4 nm, about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about or 3000 nm. The first subset and the second subset may be separated by a distance of no more than about 5 nm, about 6 nm, about 7 nm, about 8 nm, about 9 nm, about 10 nm, about 15 nm, about 20 nm, about 25 nm, about 30 nm, about 35 nm, about 40 nm, about 45 nm, about 50 nm, about 60 nm, about 70 nm, about 80 nm, about 90 nm, about 100 nm, about 110 nm, about 120 nm, about 130 nm, about 140 nm, about 150 nm, about 160 nm, about 170 nm, about 180 nm, about 190 nm, about 200 nm, about 250 nm, about 300 nm, about 350 nm, about 400 nm, about 450 nm, about 500 nm, about 550 nm, about 600 nm, about 650 nm, about 700 nm, about 750 nm, about 800 nm, about 850 nm, about 900 nm, about 950 nm, about 1000 nm, about 1100 nm, about 1200 nm, about 1300 nm, about 1400 nm, about 1500 nm, about 1600 nm, about 1700 nm, about 1800 nm, about 1900 nm, about 2000 nm, about 2100 nm, about 2200 nm, about 2300 nm, about 2400 nm, about 2500 nm, about 2600 nm, about 2700 nm, about 2800 nm, about 2900 nm, about or 3000 nm. The distance may be at least about 3000 nm. The distance may be at least about 2000 nm. The distance may be at least about 1000 nm. The distance may be at least about 5 nm to at least about 3000 nm. The distance may be at least about 5 nm to at least about 2500 nm. The distance may be at least about 5 nm to at least about 2000 nm. The distance may be at least about 5 nm to at least about 1000 nm.

In some embodiments, the chips described herein comprise two or more resonators. In some embodiments, each of the two or more resonators supports one or more guided modes. In some embodiments, each of the two or more resonators has a corresponding longitudinal perturbation, wherein an incident light is coupled to two or more of the guided mode resonances by the longitudinal perturbations of the resonators. In some embodiments, each resonator comprises an electrical insulator or a semiconductor. In some embodiments, each resonator comprises at least one nanogap configured to concentrate an incident light as described herein. In some embodiments, each resonator comprises about 1, 2, 3, 4, 5, 6, 7, 8, 9, or 10 nanogaps. In some embodiments, one or more regions of high electromagnetic field intensity are localized within and in proximity to each nanogap, whereby environmental sensing is provided. In some embodiments, a resonator comprises a binding moiety that is non-specific for an analyte described herein. In some embodiments, a resonator comprises a binding moiety that is specific for an analyte described herein. In some embodiments, the resonator does not comprise a binding moiety. In some embodiments, two or more resonators of the array are parallel to one another. In some embodiments, two or more resonators of the array are nonparallel to one another. In some embodiments, each resonator further comprises a photonic crystal mirror designed to confine the guided mode resonance.

An array can have a quality factor (Q) descriptive of the efficiency of the array at concentrating electric field. A higher Q can be related to an array that more efficiently concentrates an incident light field, resulting in higher field strengths in the array. The arrays of the present disclosure can have a quality factor of at least about 100, 200, 300, 400, 500, 600, 700, 800, 900, 1,000, 2,000, 3,000, 4,000, 5,000, 7,500, 10,000, 15,000, 20,000, 25,000, 35,000, 50,000, 75,000, 100,000, or more. The arrays of the present disclosure can have a quality factor of at most about 100,000, 75,000, 50,000, 35,000, 25,000, 20,000, 15,000, 10,000, 7,500, 5,000, 4,000, 3,000, 2,000, 1,000, 900, 800, 700, 600, 500, 400, 300, 200, 100, or less. The arrays of the present disclosure can have a quality factor in a range as defined by any two of the preceding values. Such Q can be achieved in part due to the presence of the gap in the array, which can result in the enhanced field localization that causes the high Q of the array. Similarly, the reduced volume of the gap can result in reduced mode volume of the resonator. The mode volume of the resonator can be less than the wavelength of the light used to excite the resonator. The mode volume of the resonator may be at most about 2,000, 1,900, 1,800, 1,700, 1,600, 1,500, 1,400, 1,300, 1,200, 1,100, 1,000, 900, 800, 700, 600, 500, or fewer nanometers. Examples of high Q resonators and the calculations related to such resonators can be found in “Very-Large-Scale Integrated High-Q Nanoantenna Pixels (VINPix)” by Varun Dolia et. al., arXiv preprint arXiv:2310.08065 (2023), which is incorporated herein by reference in its entirety.

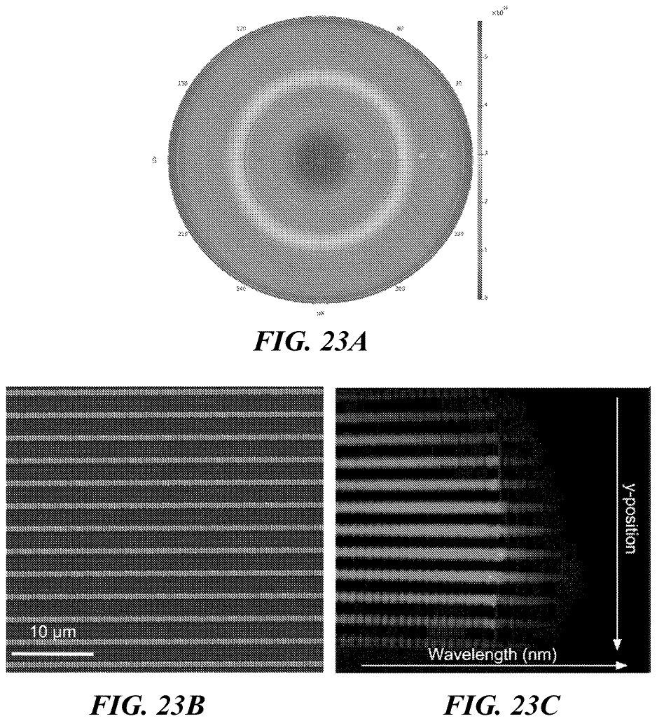

The arrays of the present disclosure may be configured to have controlled far field scattering. FIGS. 23A-23C show far field scattering profiles of arrays, according to some embodiments. As seen in FIG. 23A, the far field scattering profile of an array shows that the array is highly isotropic in its scattering profile. Additionally, the scattering is concentrated on the array itself (the center of the radial plot), showing good localization to the array. In FIGS. 23B-23C, the localization of the field to the arrays themselves can be seen. The separation of the fields can result in reduced crosstalk between the arrays and improved signal.

### (ii) Biological Samples

The systems described herein may be used for the analysis of a biological sample. In some embodiments, the sample is a liquid. In some embodiments, the sample is a dissolved solid. Though described herein with regards to biological samples, various other types of samples can be utilized with the methods and systems of the present disclosure. For example, samples from reactors can be utilized (e.g., samples used in line or batch from catalytic reactors (e.g., to generate polymers or other chemicals)). In other cases, environmental samples (e.g., water, soil, air, etc.) can be used. In other cases, food samples can be analyzed for, for example, a presence or absence of an adulterant, presence or absence of a key analyte, etc. Similarly, samples can be processed to identify low-concentration portions of the sample (e.g., in forensic samples, etc.).

The biological sample may be a tissue sample. The biological sample may be a single cell. The biological sample may be a plurality of cells. Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. The biological sample may comprise an organism, including without limitations, a bacterium or a virus. The biological sample may comprise a cell fragment. The cells may be eukaryotic. The cells may be prokaryotic.

In some cases, the biological sample is a tissue, tissue homogenate, or organoid sample. In some cases, the biological sample is a collection of cells, or a single cell, or cell fragment. In some cases, the sample comprises polynucleotides, such as DNA or RNA. In some cases, the sample comprises macromolecules, such as proteins, including antibodies. In some cases, the sample comprises polypeptides or peptides. In some cases, the sample comprises metabolites or other small molecules. In some cases, the sample may comprise one or more viruses. In some cases, the sample may comprise one or more micro-organisms, like bacteria. In some cases, the sample may comprise mixtures or conjugates of one or more of the above example samples (e.g., antibody-drug conjugates, DNA-barcoded proteins, etc). In some cases, the sample may be a mixture of molecules, or polymers, or microplastics.

In some embodiments, the sample comprises at least one component. In some embodiments, the sample comprises two or more components. In some embodiments, the sample comprises, at least 2, 3, 4, 5, 6, 7, 8, 9, 10, 11, 12, 13, 14, 15, 16, 17, 18, 19, 20 or more components.

The component may be a nucleic acid. The nucleic acid may be DNA, RNA, or a combination thereof. The nucleic acid may be an oligonucleotide. The component may be a polypeptide or a protein. The component may be a metabolite. The component may be a polymer. The component may be a microplastic.

Though described herein with regards to biological samples, other samples can be used in the methods and systems of the present disclosure. For example, non-biological samples can be analyzed for various non-biological analytes. Examples of non-biological samples include, but are not limited to, polymers, industrial chemicals, environmental samples, or the like.

### Methods for Label-Free Spectroscopy

In certain aspects, described herein are methods of using a chip as described herein. In some embodiments, the methods described herein comprise a method of processing a biological sample. In some embodiments, the method described herein comprise a method of detecting or identifying an analyte's interaction with a sample. In some embodiments, the methods described herein comprise a method of filtering a sample. In some embodiments, the methods described herein comprise a method of detecting or identifying an analyte in a sample.

In certain aspects, described herein is a method of detecting or identifying an analyte's interactions with a sample. In some embodiments, the method comprises providing the analyte on a chip described herein. The chip may comprise an array of non-uniform features, wherein a feature of said array of non-uniform features comprises an electrical insulator or a semiconductor, wherein said feature comprises a nanogap.

In certain aspects, described herein is a method of filtering a sample as described herein. In some embodiments, the method comprises providing a biological sample on a chip as described herein. The chip may comprise an array of non-uniform features configured to filter the one or more components according to charge as described herein. The chip may be interspersed with a plurality of electrodes or functionalized features configured to filter the one or more components according to charge or size. The methods may further comprise using the chip to filter the sample.

In certain aspects, described herein is a method of detecting or identifying an analytes interactions with a sample. The methods may comprise providing the analyte on a chip as described herein. The methods may comprise introducing a sample on the chip, wherein the chip comprises the analyte. The methods may then comprise following the real-time interactions of the analyte with the sample. The sample may be a sample as described herein. The sample may comprise a protein. The sample may comprise a small molecule. The analyte may comprise a protein. The analyte may comprise an enzyme. The analyte may comprise a kinase. The analyte may comprise a receptor. The analyte may comprise a tyrosine kinase. The analyte may comprise a Janus kinase 3. The analyte may comprise an epidermal growth factor receptor (EGFR).

In some embodiments, the methods comprise introducing a sample as described herein on the chip. In some embodiments, the methods comprise, exposing the chip to a first light from a light source, such that the first light interacts with said array of non-uniform features and is further concentrated in said nanogap. In some embodiments, the methods comprise detecting a second light from said array of non-uniform features subsequent to said array of non-uniform features being exposed to the first light. In some embodiments, the methods comprise collecting a time series of the second light. In some embodiments, the second light yields infrared scattering signature associated with the analyte. In some embodiments, the second light yields a vibrational scattering signature associated with the analyte. In some embodiments, the vibrational scattering signature is a Raman spectrum. In some embodiments, the second light yields autofluorescence associated with the analyte. In some embodiments, the second light is autofluorescence associated with the analyte.

In some embodiments, a light source of the first light is integrated with the chip. In some embodiments, a light source of the first light is not integrated with the chip. In some embodiments, a light source of the second light is integrated with the chip. In some embodiments, a light source of the second light is not integrated with the chip. The incident light may be an incident laser, a light emitting diode (LED) light, a lamp, or a combination thereof. The incident light may be an LED. The incident light may be a lamp. The incident light may be a laser as described herein.

In some embodiments, the methods further comprise providing a detector. The detector may comprise a detector plane. The methods may comprise using the detector to scan wavelengths in the detector plane. In some embodiments, the second light is detected with an integrated spectrometer or filter system on the chip. In some embodiments, the second light is detected with an integrated spectrometer or filter system that is not integrated with the chip.

In some embodiments, the methods further comprise imaging at least a portion of the chip. In some embodiments, the imaging comprises super resolution imaging. Super resolution imaging includes, without limitations, structured illumination microscopy (SIM), entropy based super resolution imaging (ESI), stochastic optical reconstruction microscopy (STORM), super resolution optical fluctuation imaging (SOFI), and stimulated emission depletion microscopy (STED). In some embodiments, the super resolution imaging comprises SIM. In some embodiments, the super resolution imaging comprises ESI. In some embodiments, the super resolution imaging comprises STORM. In some embodiments, the super resolution imaging comprises SOFI. In some embodiments, the super resolution imaging comprises STED.

In some embodiments, the methods described herein further comprise producing one or more hyperspectral images. In some embodiments, each of the one of more hyperspectral images represents a distinct Raman signature. In some embodiments, the methods described herein further comprise collecting data from the one or more hyperspectral images. In some embodiments, the method further comprises scanning a biological sample to produce the hyperspectral image. The scanning may comprise a spatial scanning, spectral scanning, non-scanning, spatiospectral scanning, or any combination thereof.

Various methods of detection can be utilized with the methods and systems of the present disclosure. Examples of detection schemes include, but are not limited to, spontaneous Raman spectroscopy, Stimulated Raman spectroscopy, coherent anti-Stokes Raman spectroscopy, hyperspectral mapping using spectral filters on the detection side of the sample, hyperspectral mapping using a fixed pump laser and a variable probe wavelength, super resolution Raman imaging (e.g., using, for example, structured illumination microscopy, entropy based super resolution imaging, stochastic optical reconstruction microscopy, super resolution optical fluctuation imaging, etc.), or the like.

In some cases, the methods and systems of the present disclosure can identify an analyte with an accuracy, sensitivity, or specificity of at least about 60, 65, 70, 75, 80, 85, 90, 91, 92, 93, 94, 95, 96, 97, 98, 99, 99.9, or more percent. In some cases, the accuracy, specificity, or sensitivity can be achieved without use of a label (e.g., in a label-free manner).

In some embodiments, the methods described herein further comprise developing a machine learning model. In some embodiments, the machine learning model is a neural network. In some embodiments, the neural network is a convolutional neural network (CNN). In some cases, the neural network is a large language model (LLM).

In certain aspects, described herein is a method of detecting or identifying an analyte in a biological sample. In some embodiments, the method comprises providing said biological sample on a chip comprising an array of non-uniform features, wherein a feature of said array of non-uniform features comprises an electrical insulator or a semiconductor, wherein said feature comprises a nanogap. In some embodiments, the method comprises exposing said chip to a first light from a light source, such that said first light interacts with said array of non-uniform features and is further concentrated in said nanogap. In some embodiments, the method comprises detecting a second light from said array of non-uniform features subsequent to said array of non-uniform features being exposed to said first light. In some embodiments, the method comprises using said second light to detect or identify said analyte.

In certain aspects, described herein is a method of filtering a sample. In some embodiments, the method comprises providing a biological sample comprising one or more components on a chip, said chip comprising an array of non-uniform features configured to filter said one or more components according to size. In some embodiments, the method comprises. wherein said array of non-uniform features are interspersed with a plurality of electrodes or functionalized features configured to filter said one or more components according to charge or size; In some embodiments, the method comprises using said chip to filter said sample comprising one or more components.

In certain aspects, described herein is a method of detecting or identifying an analyte's interactions with a sample. In certain embodiments, the method comprises providing said analyte on a chip comprising an array of non-uniform features, wherein a feature of said array of non-uniform features comprises an electrical insulator or a semiconductor, wherein said feature comprises a nanogap. In certain embodiments, the method comprises introducing a sample on said chip. In certain embodiments, the method comprises exposing said chip to a first light from a light source, such that said first light interacts with said array of non-uniform features and is further concentrated in said nanogap. In certain embodiments, the method comprises detecting a second light from said array of non-uniform features subsequent to said array of non-uniform features being exposed to said first light. In certain embodiments, the method comprises collecting a time series of the second light.

The present disclosure provides a method of detecting or identifying an analyte in a biological or chemical sample, comprising providing said biological or chemical sample on a chip comprising two or more resonators, wherein each of the two or more resonators supports one or more guided modes; wherein each of the two or more resonators has a corresponding longitudinal perturbation, where at least one guided mode resonance is supported in each resonator; wherein an incident light is coupled to two or more of the guided mode resonances by the longitudinal perturbations of the resonators; a feature of said array of non-uniform features wherein each resonator comprises an electrical insulator or a semiconductor; wherein each resonator comprises at least one, wherein said feature comprises a nanogap configured to concentrate an incident light; wherein one or more regions of high electromagnetic field intensity are localized within and in proximity to each nanogap, whereby environmental sensing is provided. In some embodiments, the method comprises exposing said chip to a first light from a light source, such that said first light interacts with resonators and is further concentrated in said nanogaps. In some embodiments, the method comprises detecting a second light from resonators subsequent to said array of non-uniform features being exposed to said first light. In some embodiments, the method comprises using said second light to detect or identify said analyte.

In certain aspects, described herein is a chip-based method of filtering a sample prior to detecting or identifying a analyte and/or interactions between an analyte and a binding molecule, comprising a biological or chemical sample comprising one or more components on a chip, said chip comprising an array of non-uniform features configured to filter said one or more components according to size, and wherein said array of non-uniform features are interspersed with a plurality of electrodes or functionalized features configured to filter said one or more components according to charge, or size, or chemical/biological affinity. In some cases, the filtering is on chip filtering (e.g., filtering using one or more elements of a chip). In some embodiments, the filtering is off chip filtering (e.g., filtering the analytes before the analytes are introduced to the chip). Examples of off-chip filtering include, but are not limited to, microfiltration, ultrafiltration, nanofiltration, reverse osmosis, size-exclusion chromatography, ion-exchange chromatography, affinity chromatography, liquid chromatography, high-performance liquid chromatography (HPLC), gas chromatography, paper chromatography, thin-layer chromatography, or the like, or any combination thereof. In some embodiments, the method comprises using said chip to filter said sample comprising one or more components. In some embodiments, the method comprises providing said filtered sample on a sensor region on the same chip, said sensor region comprising two or more resonators, wherein each of the two or more resonators supports one or more guided modes; wherein each of the two or more resonators has a corresponding longitudinal perturbation, where at least one guided mode resonance is supported in each resonator; wherein an incident light is coupled to two or more of the guided mode resonances by the longitudinal perturbations of the resonators; a feature of said array of non-uniform features wherein each resonator comprises an electrical insulator or a semiconductor; wherein each resonator comprises at least one, wherein said feature comprises a nanogap configured to concentrate an incident light; wherein one or more regions of high electromagnetic field intensity are localized within and in proximity to each nanogap, whereby environmental sensing is provided. In some embodiments, the method comprises exposing said sensor region to a first light from a light source, such that said first light interacts with resonators and is further concentrated in said nanogaps. In some embodiments, the method comprises detecting a second light from resonators subsequent to said array of non-uniform features being exposed to said first light. In some embodiments, the method comprises using said second light to detect or identify said analyte.

In certain aspects, described herein is a method of detecting or identifying an analyte's interactions with a sample. In some embodiments, the method comprises providing said analyte on a chip comprising two or more resonators, wherein each of the two or more resonators supports one or more guided modes; wherein each of the two or more resonators has a corresponding longitudinal perturbation, where at least one guided mode resonance is supported in each resonator; wherein an incident light is coupled to two or more of the guided mode resonances by the longitudinal perturbations of the resonators; a feature of said array of non-uniform features wherein each resonator comprises an electrical insulator or a semiconductor; wherein each resonator comprises at least one, wherein said feature comprises a nanogap configured to concentrate an incident light; wherein one or more regions of high electromagnetic field intensity are localized within and in proximity to each nanogap, whereby environmental sensing is provided. In some embodiments, the method comprises introducing a sample on said chip. In some embodiments, the method comprises exposing said chip to a first light from a light source, such that said first light interacts with said array of resonators. In some embodiments, the method comprises detecting a second light from resonators subsequent to said resonators being exposed to said first light. In some embodiments, the method comprises collecting a time series of the second light.

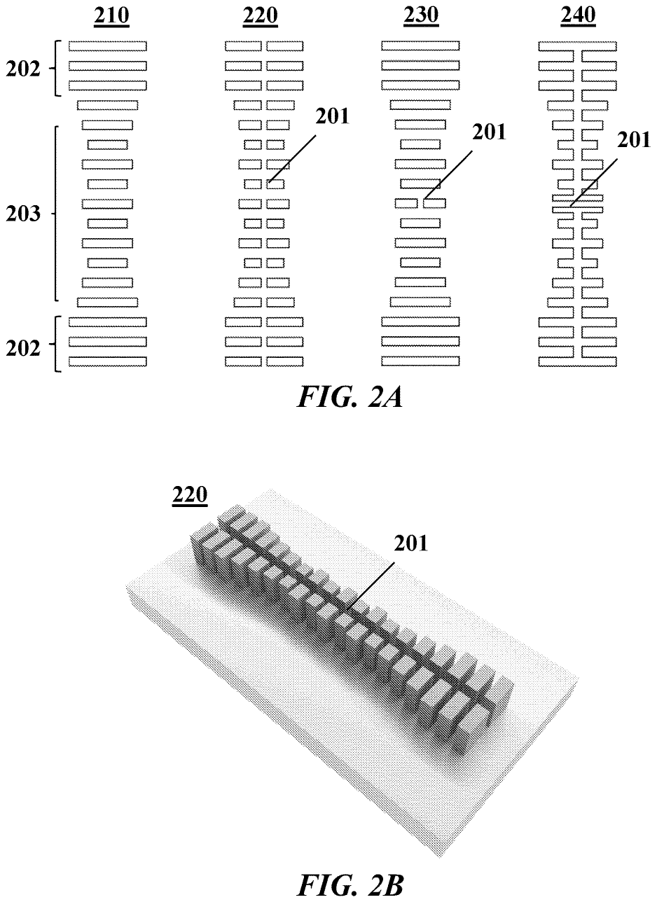

FIG. 2A shows a plurality of example designs of resonators 210, 220, 230, and 240, according to some embodiments. The resonators/arrays may be as described elsewhere herein. For example, the arrays may comprise one or more insulating materials. In another example, the arrays may comprise one or more nanogaps. Designs 220, 230, and 240 may comprise one or more nanogaps 201. The nanogaps may be as described elsewhere herein. For example, the nanogaps can be configured to concentrate an optical field within the nanogap. The array can comprise one or more regions 202 configured as photonic mirror structures. The photonic mirror structures can be configured to couple far field light into the array. The array can comprise a cavity structure 203. The cavity structure can be configured to concentrate the field into the nanogap or slot as described elsewhere herein. FIG. 2B shows an example of an alternative view of design 220, according to some embodiments. As described elsewhere herein, the individual features of the arrays may comprise features with a height, width, and length independently selected from one another. The height, width, or length may be at least about 1 nanometer (nm), 5 nm, 10 nm, 25 nm, 50 nm, 75 nm, 150 nm, 200 nm 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 micrometer (μm), 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, or more. The height, width, or length may be at most about 950 micrometers (μm), 900 μm, 850 μm, 800 μm, 750 μm, 700 μm, 650 μm, 600 μm, 550 μm, 500 μm, 450 μm, 400 μm, 350 μm, 300 μm, 250 μm, 200 μm, 150 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, 9 μm, 8 μm, 7 μm, 6 μm, 5 μm, 4 μm, 3 μm, 2 μm, 1 μm, 950 nanometers (nm), 900 nm, 850 nm, 800 nm, 750 nm, 700 nm, 650 nm, 600 nm, 550 nm, 500 nm, 450 nm, 400 nm, 350 nm, 300 nm, 250 nm, 200 nm, 150 nm, 100 nm, 75 nm, 50 nm, 25 nm, 10 nm, 1 nm, or less. The features may be separated by a distance of at least about 1 nanometer (nm), 5 nm, 10 nm, 25 nm, 50 nm, 75 nm, 150 nm, 200 nm 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 micrometer (μm), 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, or more. The features may be separated by a distance (e.g., have a gap distance or slot size) of at most about 950 micrometers (μm), 900 μm, 850 μm, 800 μm, 750 μm, 700 μm, 650 μm, 600 μm, 550 μm, 500 μm, 450 μm, 400 μm, 350 μm, 300 μm, 250 μm, 200 μm, 150 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, 9 μm, 8 μm, 7 μm, 6 μm, 5 μm, 4 μm, 3 μm, 2 μm, 1 μm, 950 nanometers (nm), 900 nm, 850 nm, 800 nm, 750 nm, 700 nm, 650 nm, 600 nm, 550 nm, 500 nm, 450 nm, 400 nm, 350 nm, 300 nm, 250 nm, 200 nm, 150 nm, 100 nm, 75 nm, 50 nm, 25 nm, 10 nm, 1 nm, or less.

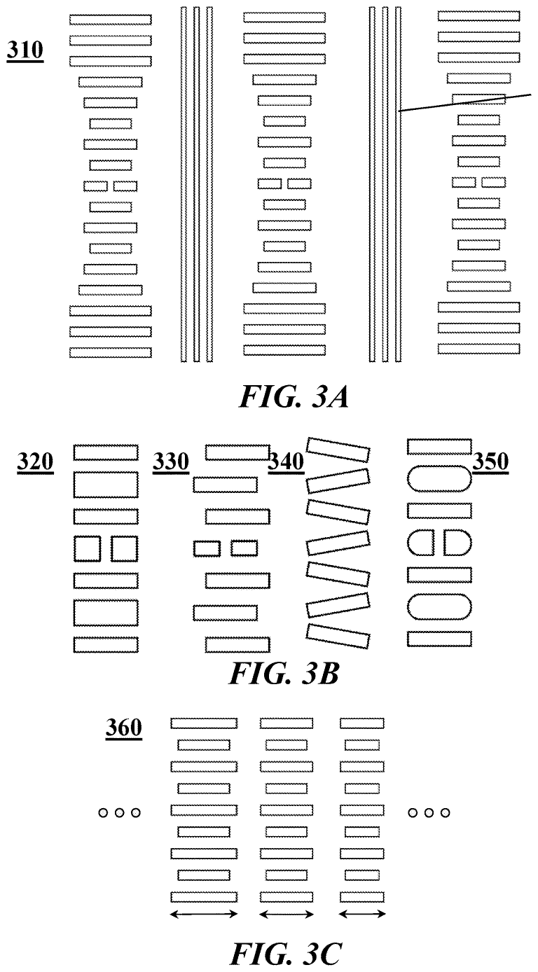

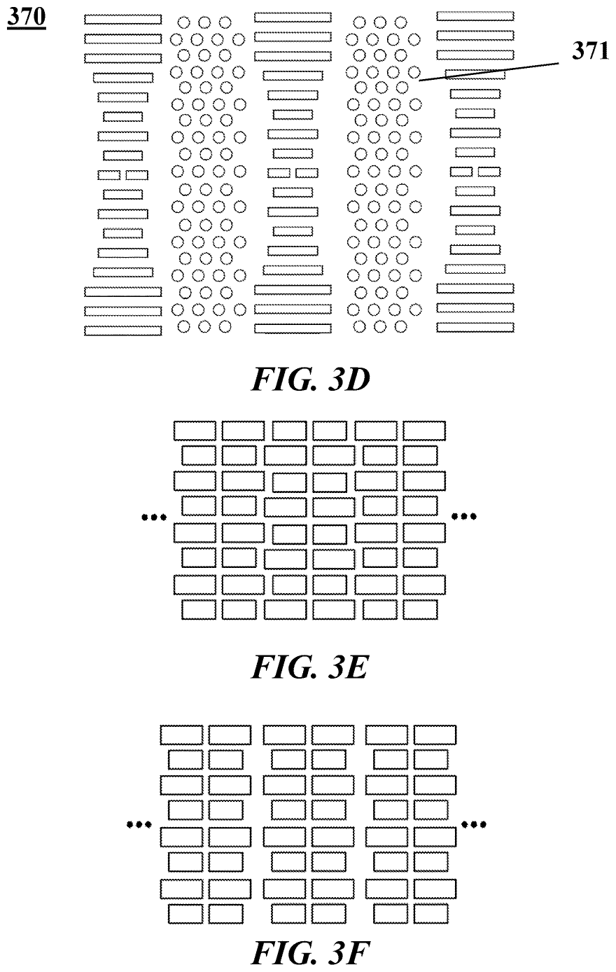

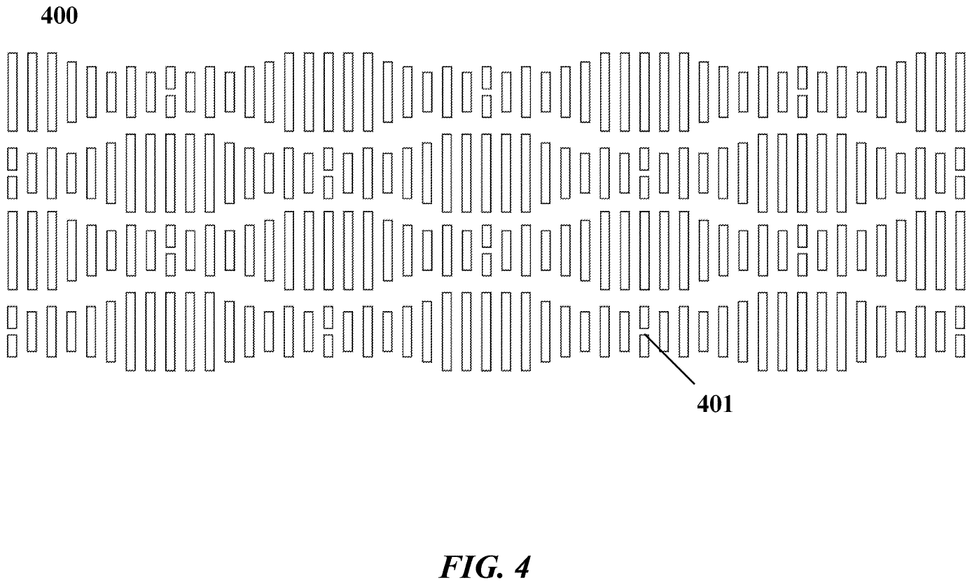

FIGS. 3A-3F show alternative designs for arrays of non-uniform features, according to some embodiments. Array 310 of FIG. 3A shows a plurality of arrays of non-uniform features placed between features 311. The features 311 may be configured to decouple the modes of the arrays. For example, the individual sensing arrays can be configured to detect a biological molecule as described elsewhere herein, and the features can reduce or eliminate the crosstalk (e.g., reduce leakage of the light fields between the individual sensing arrays). The features 311 may comprise one or more dielectric materials. The features may comprise at least about 1, 2, 3, 4, 5, 6, 7, 8, 9, 10, or more features. The features may comprise at most about 10, 9, 8, 7, 6, 5, 4, 3, 2, or fewer features. The sensing arrays may be as described elsewhere herein. For example, the sensing arrays may comprise one or more nanogaps. The presence of the features 311 may enable close spacing of the sensing arrays. For example, the presence of the features can enable reduced cross talk in arrays spaced less than about 2 micrometers from one another. FIG. 3B shows a plurality of example designs of arrays 320, 330, 340, and 350 of non-uniform features. The various designs provide different perturbations can provide different fields. The perturbations can provide benefits such as, for example, decoupling adjacent array's fields, adjusting the field concentration, providing different coupling profiles, or the like. The perturbations may be perturbations or modulations of the dimensions, positions, angles, shapes, heights, or the like, of the features of the array.

FIG. 3C shows an example of an array of non-uniform features geometrically configured to reduce cross talk, according to some embodiments. The widths of the array of non-uniform features can be adjusted to decouple the modes carried by each of the arrays. For example, the width of a first array can be set such that the wavelength of light that the first array is configured to interact with is different from the wavelength of light a second adjacent array is configured to interact with. In this example, the two arrays can be positioned within a wavelength of one another without interaction of the light between the two arrays. In this example, less than one micrometer spacings can be achieved without interference between the arrays. The arrays may comprise photonic crystal mirrors. The photonic crystal mirrors may be configured to couple incident light into the resonant modes of the array. For example, the ends of a one-dimensional array may be configured as photonic crystal mirrors. The arrays may be periodic arrays. For example, the arrays can have a repeating structure (e.g., a pattern to the dimensions of the elements of the arrays). The arrays may be aperiodic (e.g., without repeating structure). The periodicity or aperiodicity may be in the width of the arrays. For example, an aperiodic array can have elements of the same height but different widths.

FIG. 3D shows an alternative set of isolating features 371, according to some embodiments. The isolating features can serve a similar role as features 311 of FIG. 3A (e.g., isolating arrays from one another). FIGS. 3E and 3F show alternate periodic array structures, according to some embodiments. The different resonators of the arrays can be interleaved such as the resonators of FIG. 3E or spatially distinct such as the resonators of FIG. 3F.

FIG. 4 shows an example of a two-dimensional array of non-uniform features 400, according to some embodiments. The array may comprise one or more gaps 401. The gaps may be as described elsewhere herein. For example, the gaps can be configured to concentrate a light field within the gap. The two-dimensional array can be configured to provide a plurality of sensing regions in a small footprint by increasing the density of gaps that can be achieved. For example, the offset nature of the gaps of the array 400 can provide individual sensing regions configured for minimal interference while reducing spacings between the gaps. Any of the arrays of the present disclosure may be suitable for use in a two-dimensional array. For example, any of the arrays of FIGS. 2-3 can be configured as a two-dimensional array. The arrays may be separated by a distance of at least about 1 nanometer (nm), 5 nm, 10 nm, 25 nm, 50 nm, 75 nm, 150 nm, 200 nm 250 nm, 300 nm, 350 nm, 400 nm, 450 nm, 500 nm, 550 nm, 600 nm, 650 nm, 700 nm, 750 nm, 800 nm, 850 nm, 900 nm, 950 nm, 1 micrometer (μm), 2 μm, 3 μm, 4 μm, 5 μm, 6 μm, 7 μm, 8 μm, 9 μm, 10 μm, 15 μm, 20 μm, 25 μm, 30 μm, 35 μm, 40 μm, 45 μm, 50 μm, 55 μm, 60 μm, 65 μm, 70 μm, 75 μm, 80 μm, 85 μm, 90 μm, 95 μm, 100 μm, 150 μm, 200 μm, 250 μm, 300 μm, 350 μm, 400 μm, 450 μm, 500 μm, 550 μm, 600 μm, 650 μm, 700 μm, 750 μm, 800 μm, 850 μm, 900 μm, 950 μm, or more. The arrays may be separated by a distance of at most about 950 micrometers (μm), 900 μm, 850 μm, 800 μm, 750 μm, 700 μm, 650 μm, 600 μm, 550 μm, 500 μm, 450 μm, 400 μm, 350 μm, 300 μm, 250 μm, 200 μm, 150 μm, 100 μm, 95 μm, 90 μm, 85 μm, 80 μm, 75 μm, 70 μm, 65 μm, 60 μm, 55 μm, 50 μm, 45 μm, 40 μm, 35 μm, 30 μm, 25 μm, 20 μm, 15 μm, 10 μm, 9 μm, 8 μm, 7 μm, 6 μm, 5 μm, 4 μm, 3 μm, 2 μm, 1 μm, 950 nanometers (nm), 900 nm, 850 nm, 800 nm, 750 nm, 700 nm, 650 nm, 600 nm, 550 nm, 500 nm, 450 nm, 400 nm, 350 nm, 300 nm, 250 nm, 200 nm, 150 nm, 100 nm, 75 nm, 50 nm, 25 nm, 10 nm, 1 nm, or less.

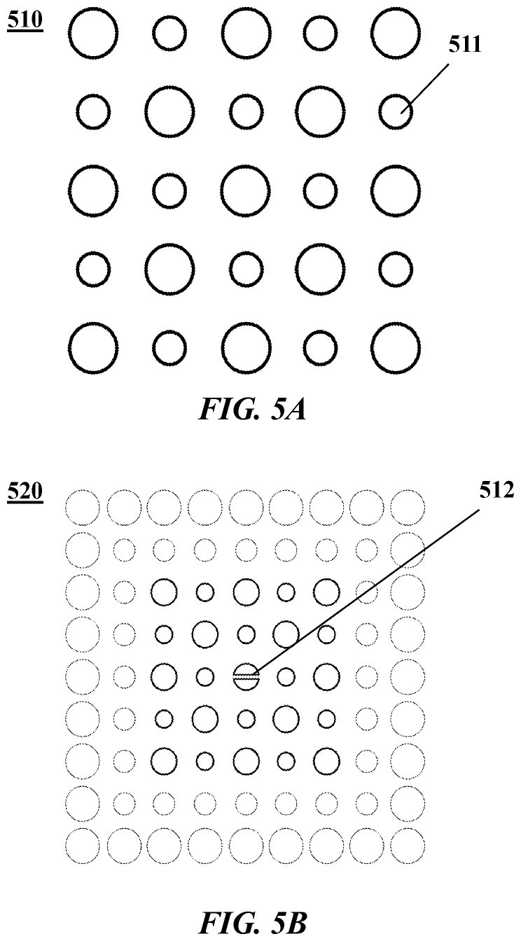

FIGS. 5A-5B show examples of two-dimensional arrays 510 and 520, according to some embodiments. The two-dimensional arrays may comprise a plurality of features 511. The features may comprise shapes such as, for example, circles, polygons (e.g., triangles, squares, rectangles, trapezoids, diamonds, pentagons, etc.), lines, or the like, or any combination thereof. In the example of FIGS. 5A-5B, the features can be circles. The features can be configured to concentrate light fields as described elsewhere herein. For example, the array 510 can be an alternative to array 210 of FIG. 2A. As such, the arrays of the present disclosure may comprise any variation of the shape of the elements of the arrays. The array 520 can be configured with a gap 512. The gap may be as described elsewhere herein. For example, the gap can be functionalized with a capture probe for immobilizing a biological molecule within the gap. The gap may be a nanogap.

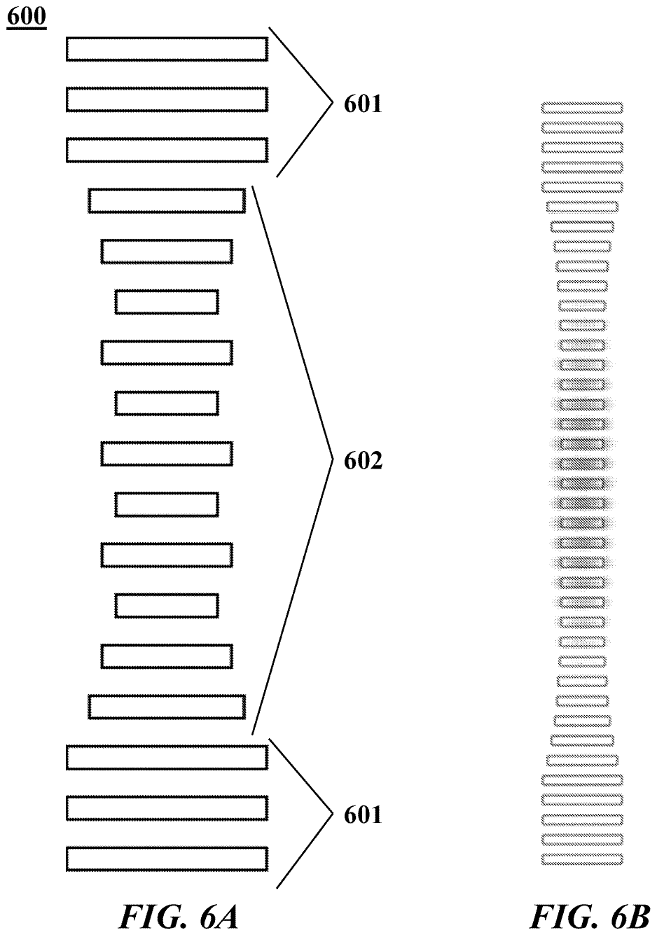

FIG. 6A shows the portions of an array 600, according to some embodiments. The array may comprise one or more photonic mirror structures 601. The photonic mirror structures may be configured to couple light into the cavity structure 602. The array may comprise at least about 1, 2, 3, 4, 5, or more photonic mirror structures. The array may comprise at most about 5, 4, 3, 2, or 1 photonic mirror structures. The photonic mirror structures may be adjusted depending on the light the photonic mirror structure is configured to interact with. For example, the photonic mirror structure can be increased in size to interact with longer wavelengths of light. The cavity structure 602 may be configured to concentrate the light coupled by the photonic mirror structure as described elsewhere herein. For example, the cavity structure may comprise a gap. FIG. 6B is an example of the field enhancement of an array not comprising a nanogap, according to some embodiments. The field enhancement may be generated by a full field simulation of the modes of the structure. In the example of FIG. 6B, an electric field enhancement of 2,500 times may be observed, which can provide a Q of the cavity of about 80,000. The arrays of the present disclosure can provide electric field enhancements of at least about 10, 50, 100, 200, 300, 400, 500, 600, 700, 800, 900, 1,000, 1,100, 1,200, 1,300, 1,400, 1,500, 1,600, 1,700, 1,800, 1,900, 2,000, 2,100, 2,200, 2,300, 2,400, 2,500, 2,600, 2,700, 2,800, 2,900, 3,000, 3,500, 4,000, 4,500, 5,000, 6,000, 7,000, 8,000, 9,000, 10,000, or more times. The arrays of the present disclosure can provide electric field enhancements of at most about 10,000, 9,000, 8,000, 7,000, 6,000, 5,000, 4,500, 4,000, 3,500, 3,000, 2,900, 2,800, 2,700, 2,600, 2,500, 2,400, 2,300, 2,200, 2,100, 2,000, 1,900, 1,800, 1,700, 1,600, 1,500, 1,400, 1,300, 1,200, 1,100, 1,000, 900, 800, 700, 600, 500, 400, 300, 200, 100, 50, 10, or less times.

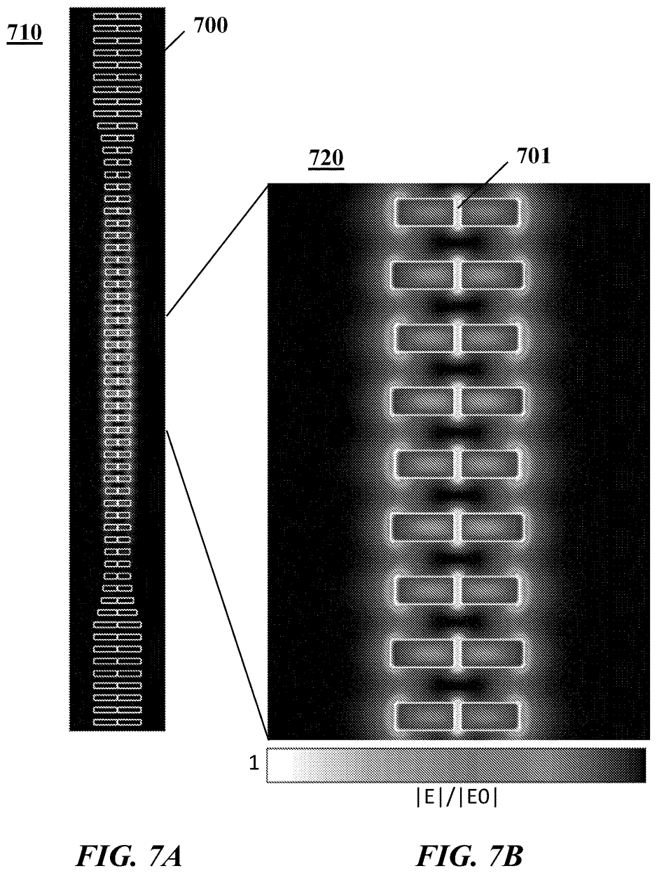

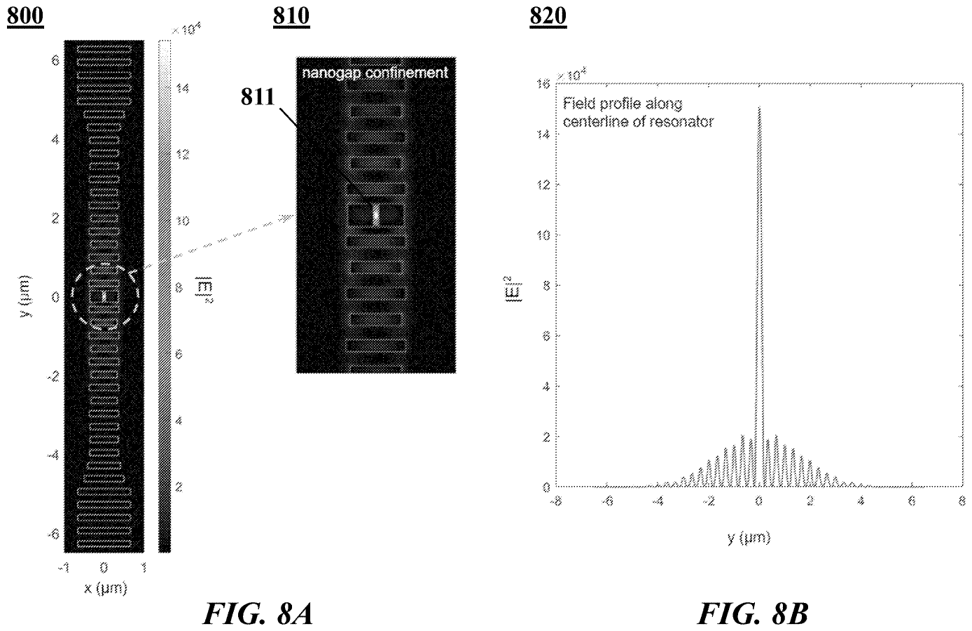

FIGS. 7A-7B show an example of a field enhancement calculation 710 and an inset zoom 720 of an array 700 comprising a plurality of gaps 701, according to some embodiments. As described elsewhere herein, the plurality of gaps can be configured to concentrate a field of an incident light within the gaps. As can be seen in the calculations, the field can be largely confined to the gaps, thereby improving the signal that can be achieved. In the example of FIGS. 7A-7B, the array can comprise a plurality of nanogaps. In some cases, each member of the array can comprise at least one nanogap. Each nanogap of the plurality of nanogaps can be functionalized as described elsewhere herein. For example, each nanogap can be configured with agents configured to bind one or more analytes. In some cases, an array can comprise a single nanogap. For example, FIG. 8A shows an example of an array 800 with a single nanogap 811, according to some embodiments. The inset 810 may show the nanogap region in increased detail. The plot 820 of FIG. 8B is an example of an absolute field profile along the transverse direction of the array member comprising nanogap 811. As seen in 820, the high field confined to the center of the nanogap can provide the benefits described elsewhere herein (e.g., enhanced detection of an analyte configured within the nanogap).

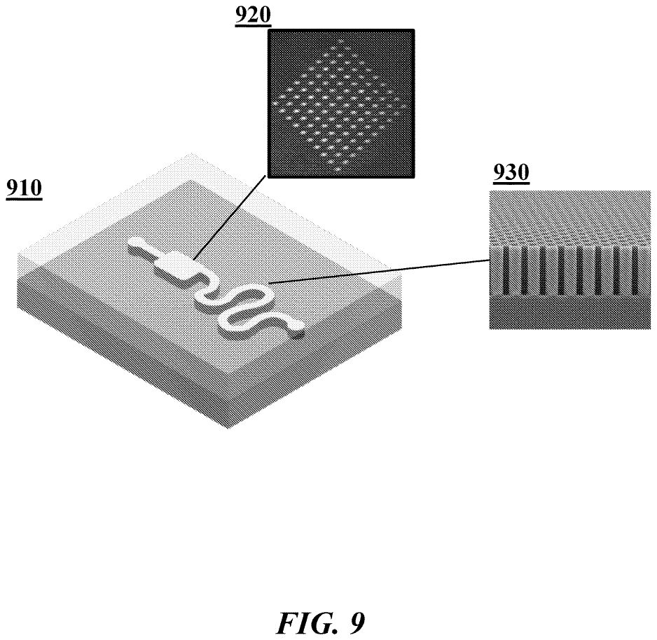

FIG. 9 shows an example of a chip 910 comprising a detection region 920 and a separation region 930, according to some embodiments. The chip may comprise at least about 1, 2, 3, 4, 5, 6, 7, 8, 9, 10, or more detection regions. The chip may comprise at least about 1, 2, 3, 4, 5, 6, 7, 8, 9, 10, or more separation regions. The detection regions may comprise one or more arrays as described elsewhere herein. For example, the detection region may comprise at least one array of features comprising a nanogap. The separation region may be as described elsewhere herein. The separation region may comprise pillars of various sizes separated by various spacings configured to separate a sample by size. The pillars can comprise metals, dielectrics, insulators, or the like, or any combination thereof. The separation region may comprise one or more electrodes. The electrodes may be interdigitated (e.g., portions of the electrodes can be interspersed between other portion of the electrodes). The electrodes may be configured to separate analytes by charge. The pillars and/or substrate below the pillars may comprise one or more surface functionalizations and/or modifications. For example, the pillars can be coated with a nucleic acid sequence configured to bind and remove a non-target nucleic acid molecule from the sample.

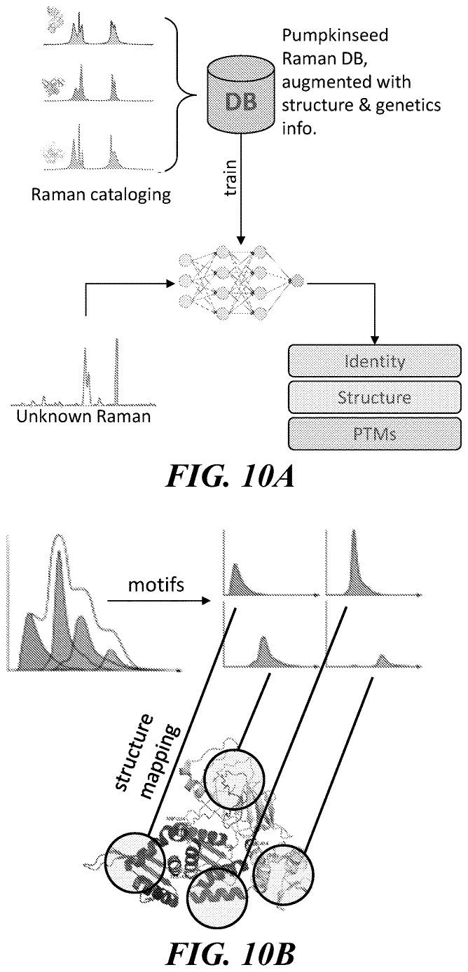

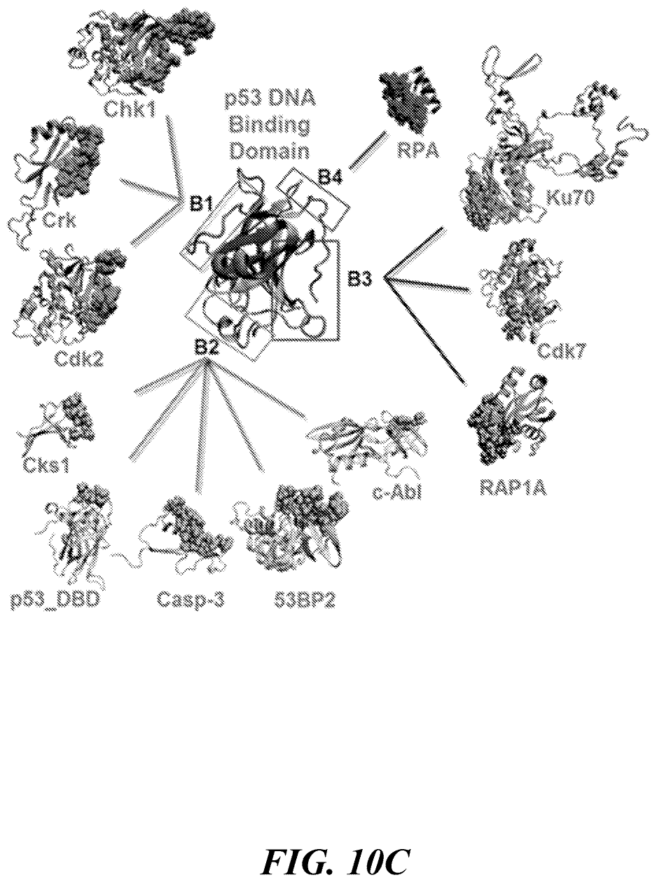

FIGS. 10A-10C show a pathway for analysis of the spectra of the present disclosure, according to some embodiments. Spectral data (e.g., Raman spectra, etc.), can be collected as described elsewhere herein and transferred to a database. The database may comprise additional information such as, for example, structure data, genetics data, sample data, or the like, or any combination thereof. The database may be used to train a machine learning algorithm. The machine learning algorithm can then be configured to analyze new data of an unknown sample to determine properties of the sample (e.g., presence or absence of analytes, structure of analytes, post-translational modifications, etc.). FIG. 10B shows an example of a structure mapping pathway, according to some embodiments. The structure mapping pathway can be configured, using one or more computer processors, to decompose a spectrum into one or more constituent signals. The constituent signals can correspond to structural motifs present in the analyte, which can provide information regarding the structural composition of the analyte. In the example of FIG. 10B, the different portions of the protein can generate Raman signals in different parts of the spectrum, which can then be used to identify the constituent portions of the analyte. In this way, recurring subunits or motifs can be identified in samples, which can provide information regarding analyte taxonomy and/or constellations. FIG. 10C shows an example of a structure prediction pathway, according to some embodiments. Using the spectra of the present disclosure, the structure of a new analyte can be predicted even if the structure is otherwise unknown. For example, based on the structure of previously determined analyte, a new analyte can be analyzed and predictions for the component structures can be made. The kinetics and/or activity of the analyte can be predicted as well (e.g., the binding kinetics of a protein). In the example of FIG. 10C, a variety of moieties are being predicted for regions B1-B4.

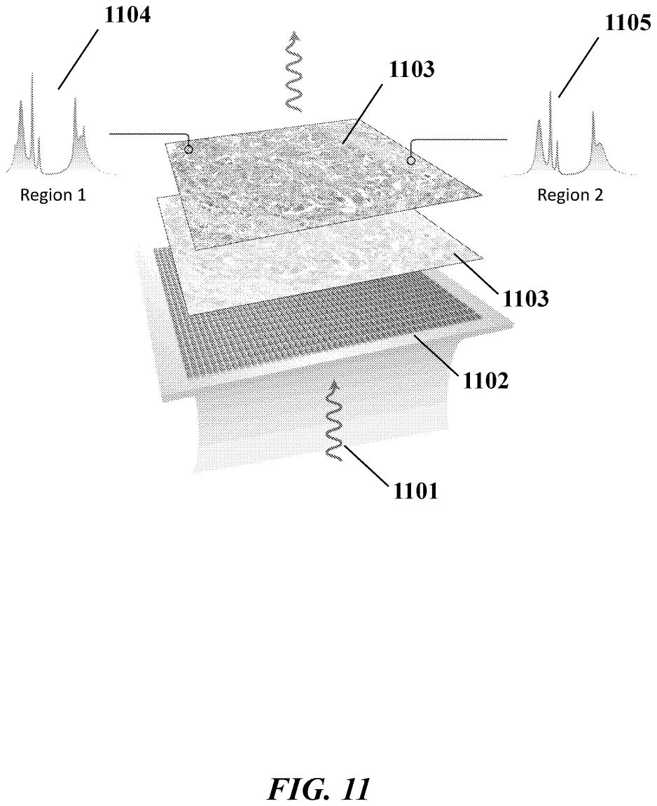

FIG. 11 shows an example of tissue mapping with an array, according to some embodiments. Light 1101 may be light as described elsewhere herein. The light can be directed from a light source (not pictured) towards an array 1102 (e.g., an array as described elsewhere herein). The array can be configured to enhance the light field within the array, which can improve the signal that the array produces. A sample 1103 can be placed adjacent to the array. For example, an unstained fixed tissue section can be placed atop an array. A plurality of samples can be placed atop an array. The sample can be placed such that portions of the sample interact with the light fields concentrated by the array. For example, the sample can be placed such that a portion of the sample is in sufficient proximity to a gap in a member of the array as to provide enhanced light fields encompassing the portion of the sample. In some cases, the array can comprise a binding moiety configured to bind to an analyte within the tissue. For example, a gap within a member of the array can comprise a nucleic acid probe configured to bind to at least a portion of an analyte comprised within the sample. In this example, the analyte can move out of the sample and be bound to the probe. The light 1101 interacting with the array 1102 and the sample 1103 can generate a hyperspectral map 1104 comprise a plurality of spectra (e.g., spectra 1104 and 1105). The hyperspectral map may provide information regarding the distribution of analytes or other features within the sample. For example, the spectra can provide identification for various analytes or structural features within the sample. In another example, the spectra can provide distribution data for analytes within the sample. The sample (e.g., analyte) may be label free. For example, the sample may not comprise a label (e.g., a fluorophore) on an analyte. For example, the sample may not be coupled to a fluorophore (e.g., not covalently coupled to the label). In this example, the hyperspectral map can provide information related to the analyte without the use of a label.

### Computer Systems

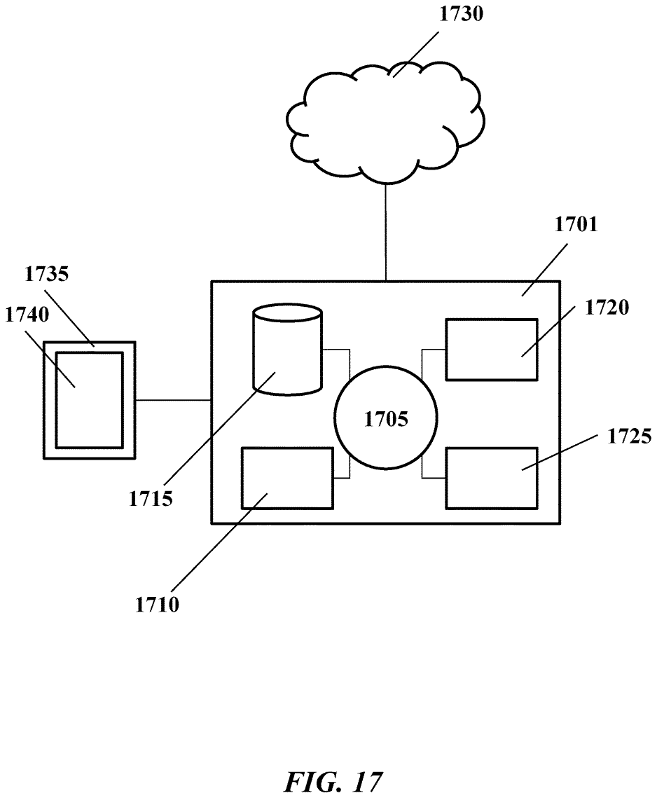

The present disclosure provides computer systems that are programmed to implement methods of the disclosure. FIG. 17 shows a computer system 1701 that is programmed or otherwise configured to implement the methods of the present disclosure. The computer system 1701 can regulate various aspects of the present disclosure, such as, for example, detection and/or analysis of Raman spectra. The computer system 1701 can be an electronic device of a user or a computer system that is remotely located with respect to the electronic device. The electronic device can be a mobile electronic device.

The computer system 1701 includes a central processing unit (CPU, also “processor” and “computer processor” herein) 1705, which can be a single core or multi core processor, or a plurality of processors for parallel processing. The computer system 1701 also includes memory or memory location 1710 (e.g., random-access memory, read-only memory, flash memory), electronic storage unit 1715 (e.g., hard disk), communication interface 1720 (e.g., network adapter) for communicating with one or more other systems, and peripheral devices 1725, such as cache, other memory, data storage and/or electronic display adapters. The memory 1710, storage unit 1715, interface 1720 and peripheral devices 1725 are in communication with the CPU 1705 through a communication bus (solid lines), such as a motherboard. The storage unit 1715 can be a data storage unit (or data repository) for storing data. The computer system 1701 can be operatively coupled to a computer network (“network”) 1730 with the aid of the communication interface 1720. The network 1730 can be the Internet, an internet and/or extranet, or an intranet and/or extranet that is in communication with the Internet. The network 1730 in some cases is a telecommunication and/or data network. The network 1730 can include one or more computer servers, which can enable distributed computing, such as cloud computing. The network 1730, in some cases with the aid of the computer system 1701, can implement a peer-to-peer network, which may enable devices coupled to the computer system 1701 to behave as a client or a server.

The CPU 1705 can execute a sequence of machine-readable instructions, which can be embodied in a program or software. The instructions may be stored in a memory location, such as the memory 1710. The instructions can be directed to the CPU 1705, which can subsequently program or otherwise configure the CPU 1705 to implement methods of the present disclosure. Examples of operations performed by the CPU 1705 can include fetch, decode, execute, and writeback.

The CPU 1705 can be part of a circuit, such as an integrated circuit. One or more other components of the system 1701 can be included in the circuit. In some cases, the circuit is an application specific integrated circuit (ASIC).

The storage unit 1715 can store files, such as drivers, libraries and saved programs. The storage unit 1715 can store user data, e.g., user preferences and user programs. The computer system 1701 in some cases can include one or more additional data storage units that are external to the computer system 1701, such as located on a remote server that is in communication with the computer system 1701 through an intranet or the Internet.

The computer system 1701 can communicate with one or more remote computer systems through the network 1730. For instance, the computer system 1701 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1701 via the network 1730.

Methods as described herein can be implemented by way of machine (e.g., computer processor) executable code stored on an electronic storage location of the computer system 1701, such as, for example, on the memory 1710 or electronic storage unit 1715. The machine executable or machine readable code can be provided in the form of software. During use, the code can be executed by the processor 1705. In some cases, the code can be retrieved from the storage unit 1715 and stored on the memory 1710 for ready access by the processor 1705. In some situations, the electronic storage unit 1715 can be precluded, and machine-executable instructions are stored on memory 1710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1701 can include or be in communication with an electronic display 1735 that comprises a user interface (UI) 1740 for providing, for example, control of Raman spectroscopy. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1705. The algorithm can, for example, analyze the Raman spectra described elsewhere herein.

The following examples are illustrative of certain systems and methods described herein and are not intended to be limiting.

### Example 1—Detection Arrays

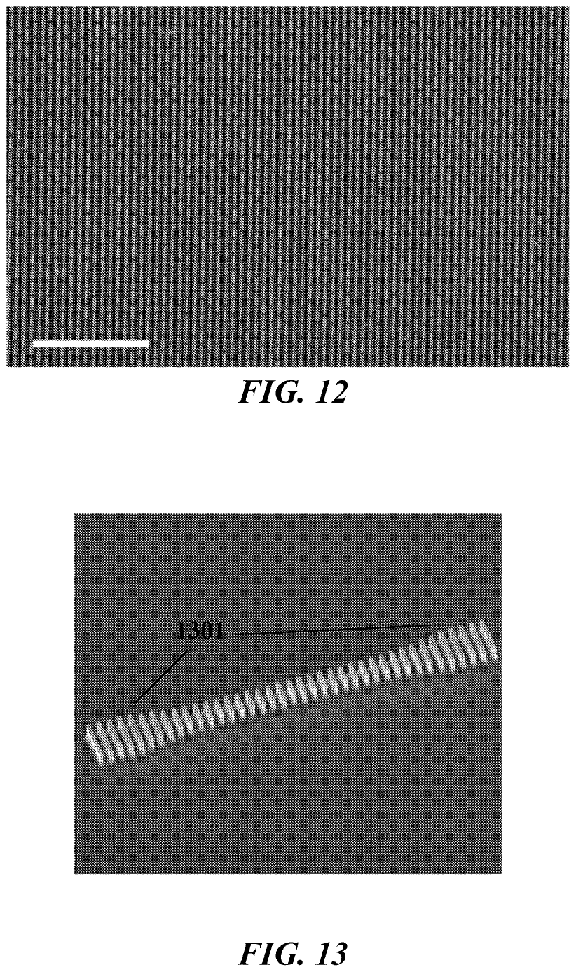

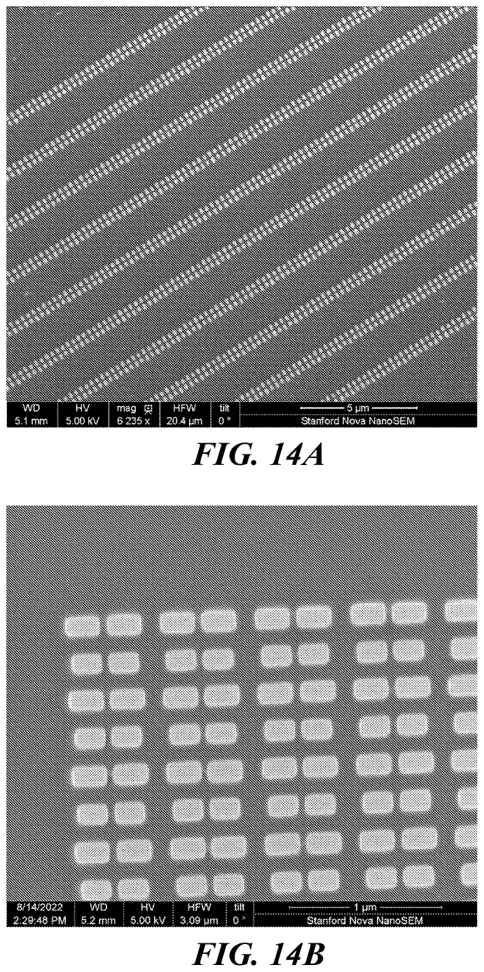

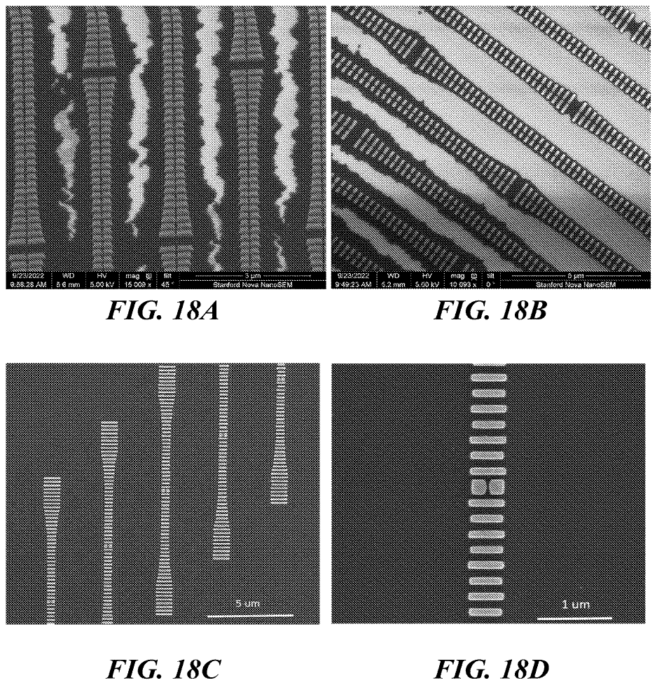

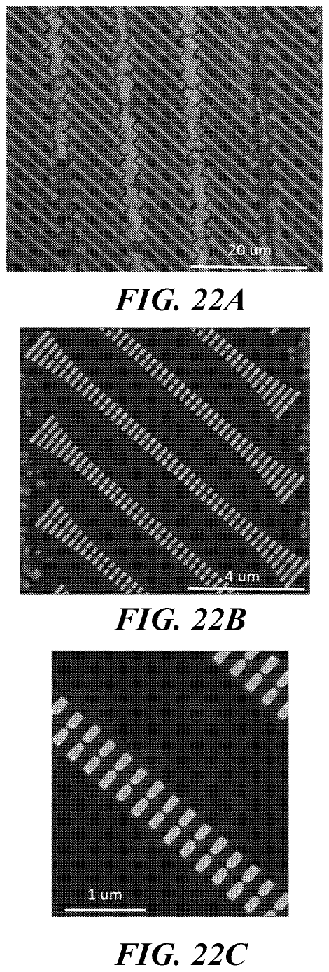

FIG. 12 is an example micrograph of a plurality of arrays, according to some embodiments. In this example, the scale bar is 200 micrometers. The plurality of arrays can be produced by, for example, lithography. In this example, the plurality of arrays has a density of three million arrays per square centimeter. Each array can be configured to detect an analyte. For example, all of the arrays can be configured to detect the same analyte. In another example, different arrays can be configured to detect different analytes. FIG. 13 is a micrograph of an example array, according to some embodiments. The array can be configured as a guided mode resonance structure, which can be configured to concentrate light and/or control far field scattering. The array may comprise one or more photonic crystal mirrors 1301 (the larger regions at the ends of the arrays). The photonic crystal mirrors can be configured to laterally confine a field (e.g., a field generated by incident light) into the guided mode resonant mode. In this way, far field incident light can be coupled into the modes of the array, which can then be concentrated as described elsewhere herein (e.g., via a gap present in a member of the array). FIGS. 14A-14B are micrographs of example pluralities of arrays comprising a plurality of gaps, according to some embodiments. The arrays can be configured to concentrate the field of incident light within the gaps, thereby providing enhanced field strength as described elsewhere herein. FIGS. 18A-18D show additional examples of fabricated arrays, according to some embodiments. The arrays can comprise a pointed portion as in FIGS. 18C and 18D. The pointed portion can be configured to further increase the field in the nanogap. Similarly, FIGS. 22A-22C show examples of fabricated arrays at different magnification levels, according to some embodiments. These arrays were fabricated with slot down the central axis of the array and with pointed portions to further enhance the field strength within the slot.

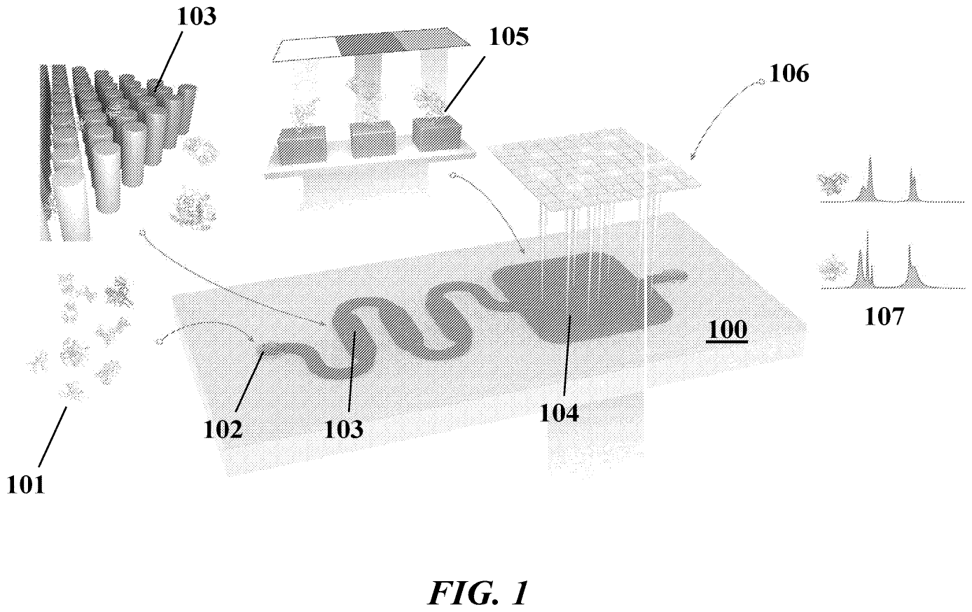

FIG. 1 shows an example of a processing workflow, according to some embodiments. A sample 101 can be introduced to a chip 100. The sample may comprise one or more molecules or components to be identified or detected. For example, a plurality of proteins can be suspended in a liquid and flowed into an inlet port 102 of the chip 100. The sample can be flowed through one or more features. The features can be as described elsewhere herein. For example, the features can be configured to filter the sample (e.g., by size, charge, etc.) to isolate analytes from the sample. The analytes can flow into the detection region 104, where the analytes can bind to capture probes 105, thereby placing the analytes into the near field of an array. The array can then be configured to concentrate an incident light field as described elsewhere herein. The interactions of the analytes with the light field can produce a signal map 106. The signal map may be produced for each analyte in parallel. For example, the same incident light beam can interact with a plurality of arrays and a plurality of analytes to generate a plurality of spectra 107 at a same time. The spectra can then be analyzed to determine the identities of the analytes as described elsewhere herein.

### Example 2—Spectral Analysis of Analytes

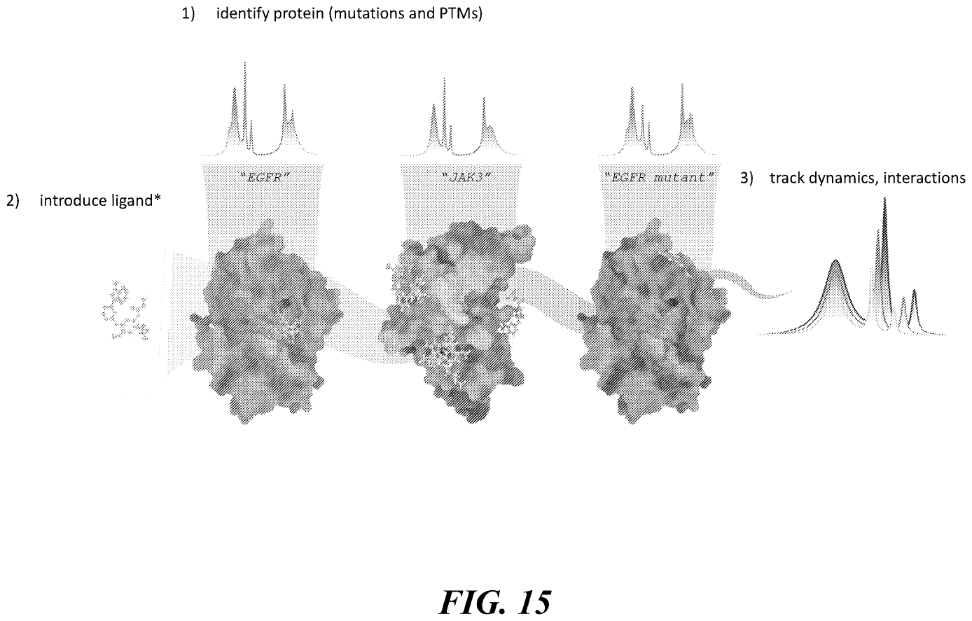

FIG. 15 shows an example of spectral measurement of an interaction, according to some embodiments. In a first operation, the identity of an analyte can be determined. In this example, protein analytes are determined to be EGFR, JAK2, and an EGFR mutant (e.g., a unique proteoform of EGFR with a prost-translational modification at one or more amino acid residues). The identity can be determined using a Raman spectrum generated by an interaction of light with the analytes within an array, as described elsewhere herein. For example, each of the analyte proteins can be bound within a gap of an array, and light can be shone onto the array to determine the Raman spectra of the analytes.

A ligand can then be introduced to the analytes. For example, a ligand can be bound to the proteins. The binding dynamics can be tracked in real time via the Raman spectra of the analytes. For example, a time series of the analytes after introduction of the ligand can be taken, and the changes in the conformation of the analytes can be tracked via the spectra.

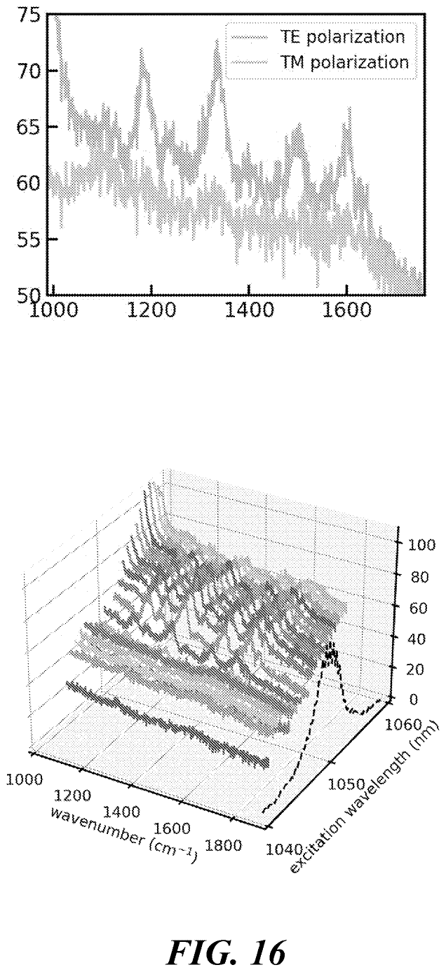

FIG. 16 shows sample Raman spectra, according to some embodiments. The Raman spectra can be generated by the methods and systems of the present disclosure. For example, an array comprising a gap can provide the Raman spectra. The arrays of the present disclosure may provide different degrees of field concentration depending on the polarity of the incident light.

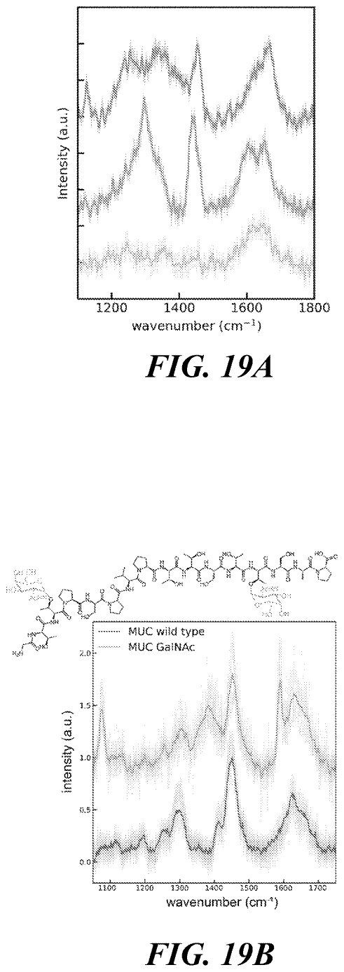

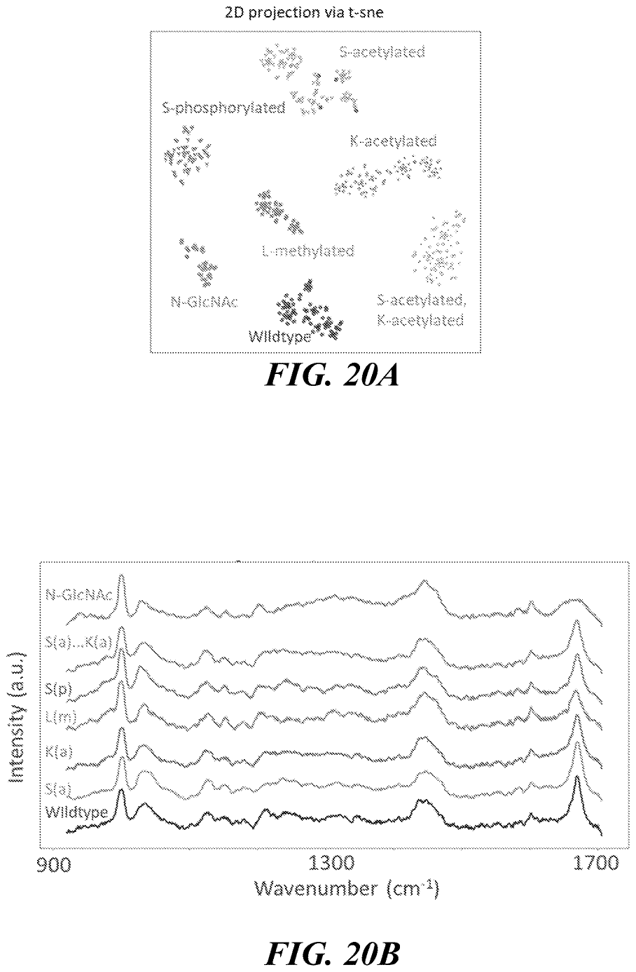

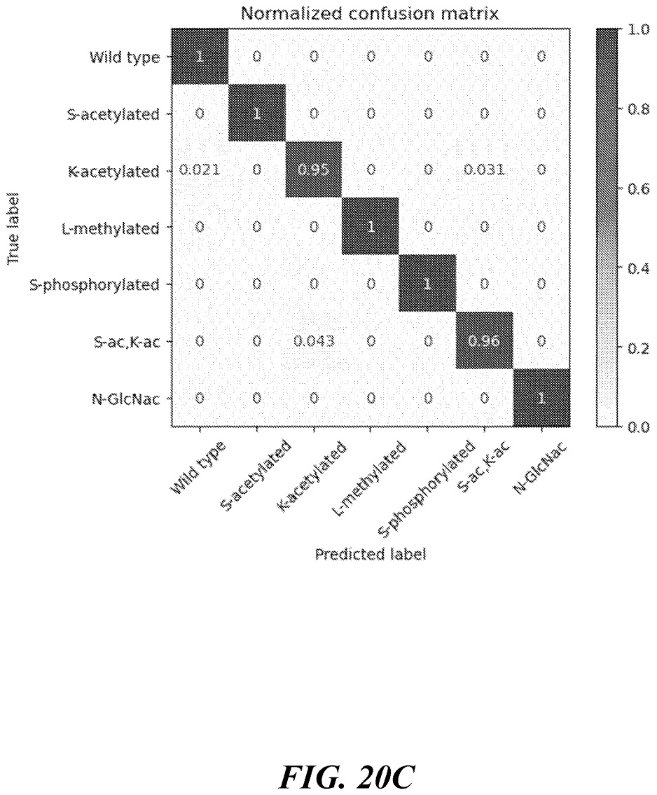

FIGS. 19A-19B show examples of Raman spectra of proteins and protein fragments according to some embodiments. The spectra may be generated from 24 attograms of material using the arrays of the present disclosure. The differences between wild type and post-translationally modified mucin protein fragments may be discernible from the Raman spectrum of FIG. 19B. FIGS. 20A-20B show examples of post translational modification spectra, according to some embodiments. FIG. 20B shows a plurality of Raman spectra of Ovalbumin epitope SIINFEKL with different post-translational modifications. The spectra can be analyzed (e.g., via machine learning) to generate the scatter plot of FIG. 20A. The scatter plot can then be used to identify the post translational modification. FIG. 20C shows a normalized confusion matrix of the data of FIG. 20A, showing the high correlation between the predicted label and the true label in the sample.

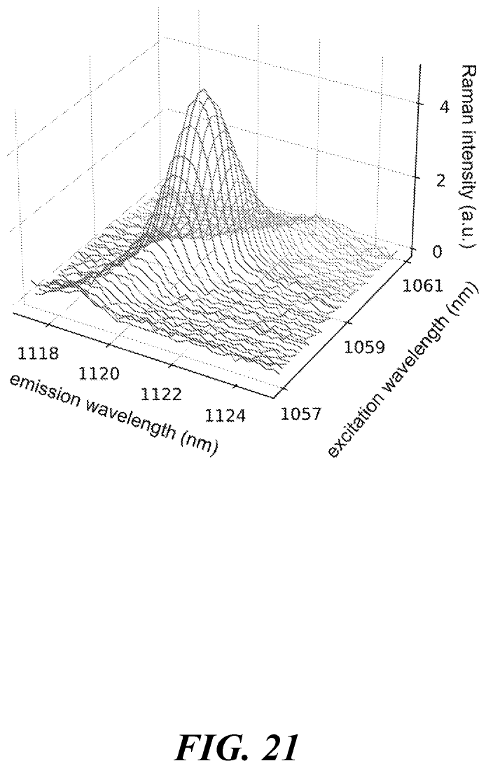

FIG. 21 shows an example of a Raman emission versus excitation wavelength plot, according to some embodiments. The plot can show that the arrays of the present disclosure may be tuned to a predetermined resonance wavelength, which can provide a high intensity at the predetermined resonance wavelength. This can, in turn, enable multiplexing or multiple wavelengths to be directed to a chip comprising a plurality of arrays each configured to be resonant with a different illumination wavelength.

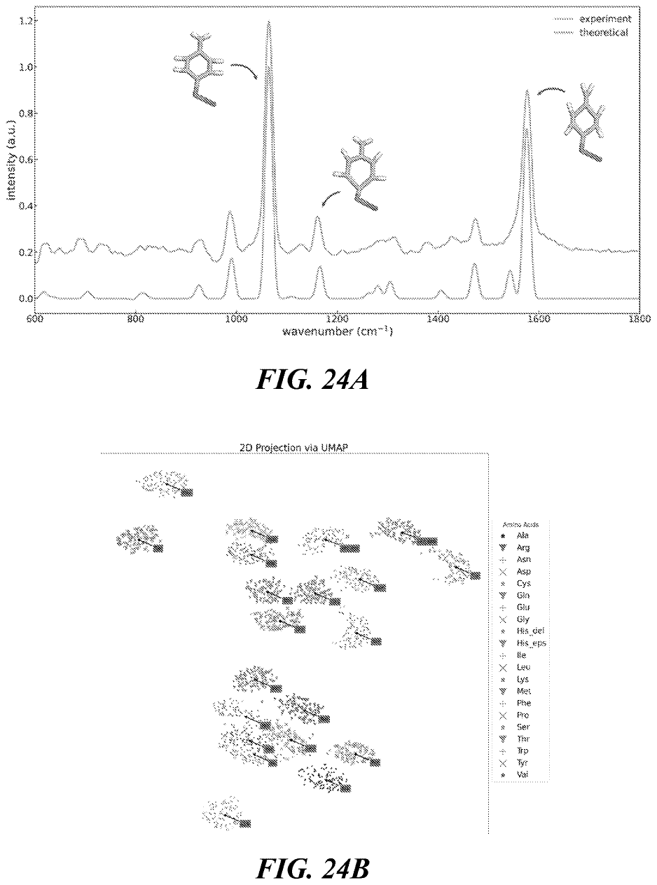

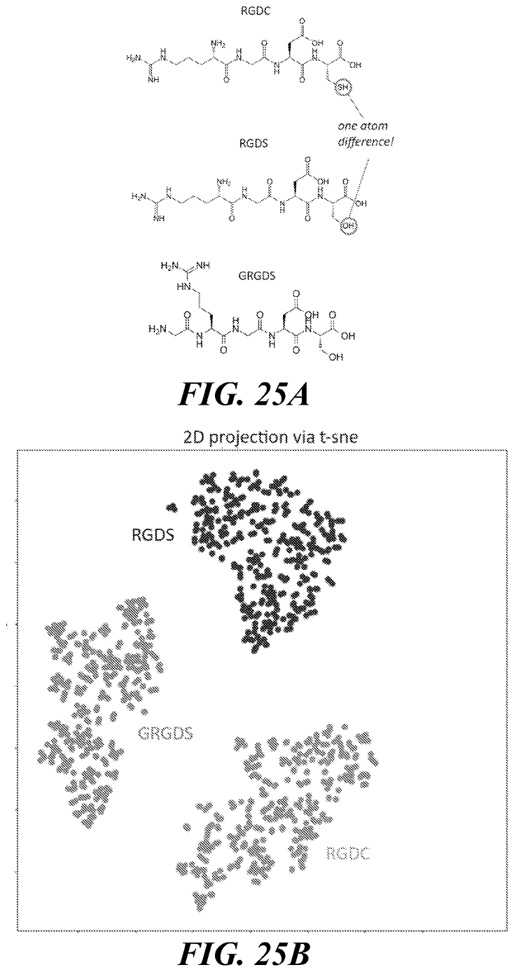

FIGS. 24A-24B show examples of simulated clustered analysis of analytes and the associated simulated Raman spectra, according to some embodiments. FIG. 24A shows an example of the strong correlation between the experimentally derived (e.g., using the methods and systems of the present disclosure) spectrum and an ab-initio calculated spectrum. Using the same model, the Raman spectra of a plurality of different amino acids were simulated at varying degrees of noise and analyzed by a clustering algorithm as described elsewhere herein. As seen in FIG. 24B, the various amino acids show strong clustering when analyzed by the algorithm, showing the ability of the methods and systems of the present disclosure to discern the different amino acids. Similarly, FIGS. 25A-25B show an example of a cluster analysis of a plurality of Raman spectra to identify analytes, according to some embodiments. Despite arginine-glycine-aspartic acid-serine (RGDS) and arginine-glycine-aspartic acid-cysteine (RGDC) differing in composition by a single atom, the classifier was able to discern the difference with 100% accuracy. Similarly, both were identified as different from glycine-arginine-glycine-aspartic acid-serine (GRGDS).

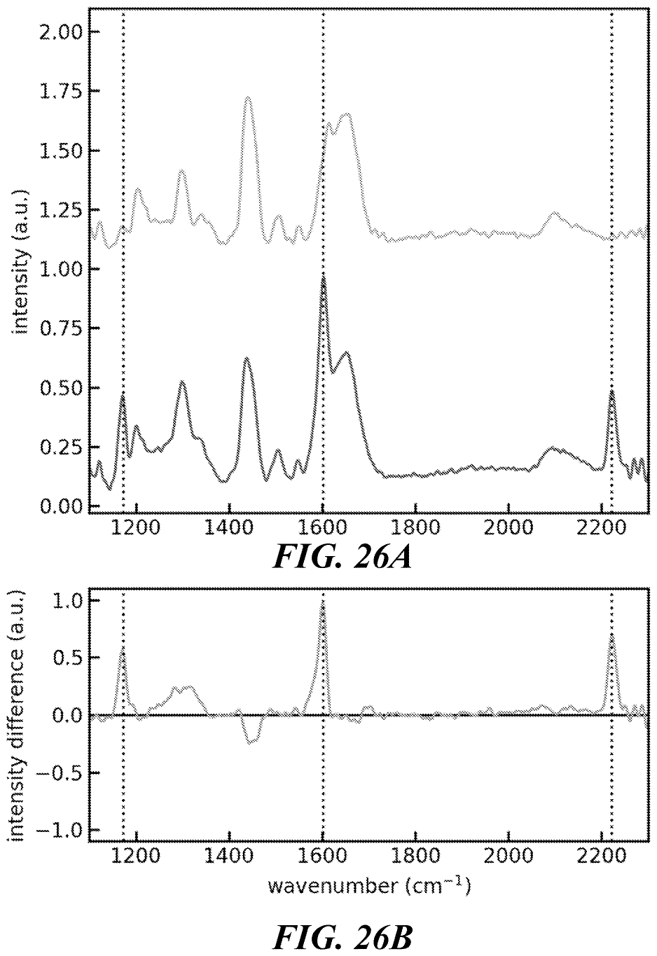

FIGS. 26A-26B show an example of Raman spectra and a difference spectrum associated with the introduction of a small molecule to a peptide, according to some embodiments. The topmost Raman spectrum of FIG. 26A shows a AYLGYLAML peptide alone, as noted in standard one letter code for each amino acid, while the bottom spectrum shows the same peptide with a trifluoroacetic acid (TFA) and phenylisothiocyanate PITC additive. The changes in the molecular structure of the peptide result in the observed difference spectrum in FIG. 26B. This shows the ability of the methods and systems of the present disclosure to differentiate small changes in peptides or other analytes. A similar process can be used to discern, for example, protein-protein interactions, protein-ligand interactions, antibody-drug conjugate interactions, etc.

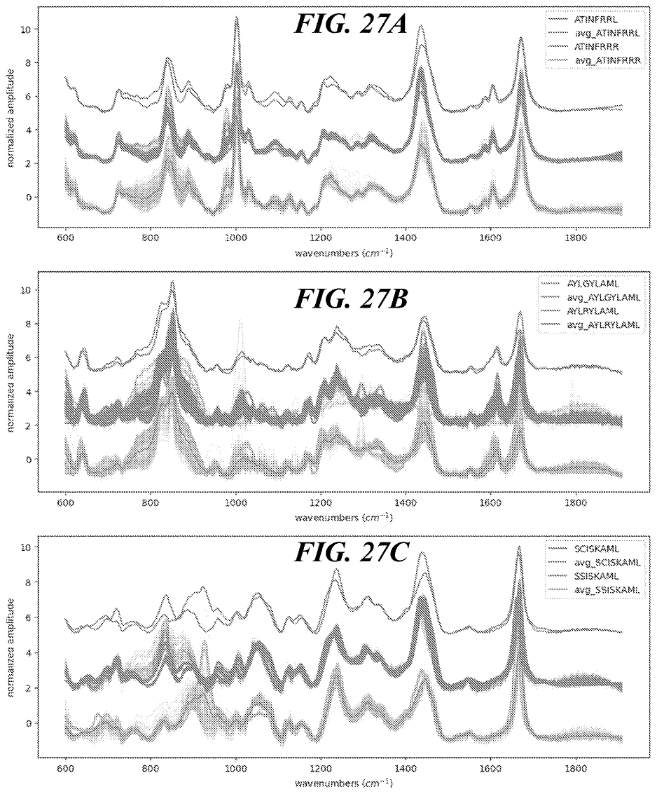

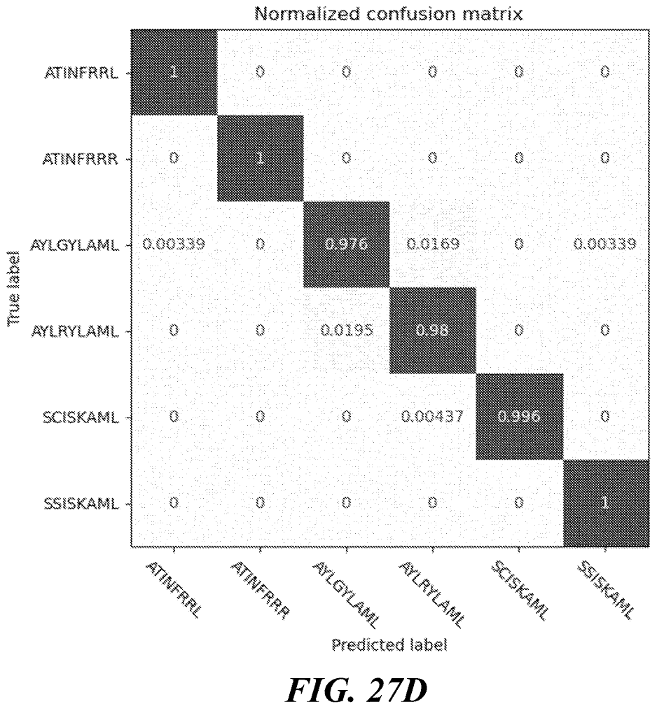

FIGS. 27A-27D show an example of a confusion matrix and associated Raman spectra, according to some embodiments. FIGS. 27A-27C show Raman spectra of three pairs of wild type versus single amino acid mutated major histocompatibility complex (MHC) peptides, as noted in standard one letter code for each amino acid, ATINFRRL vs ATINFRRR, AYLGYLAML vs AYLRYLAML, SCISKAML vs SSISKAML, respectively. The classification model was able to discern the various wild type and mutated with high accuracy, showing sensitivity to minor perturbations in the structure of the analytes.

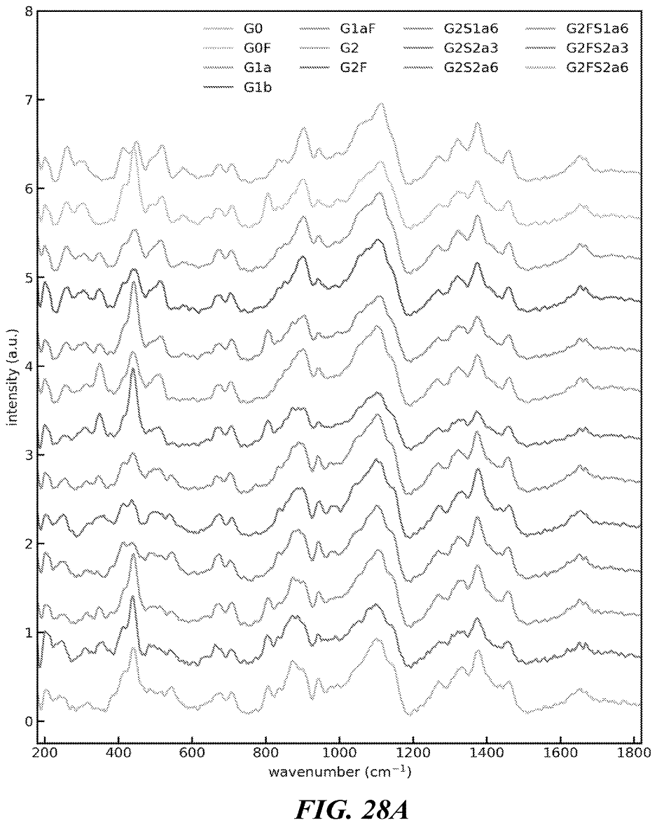

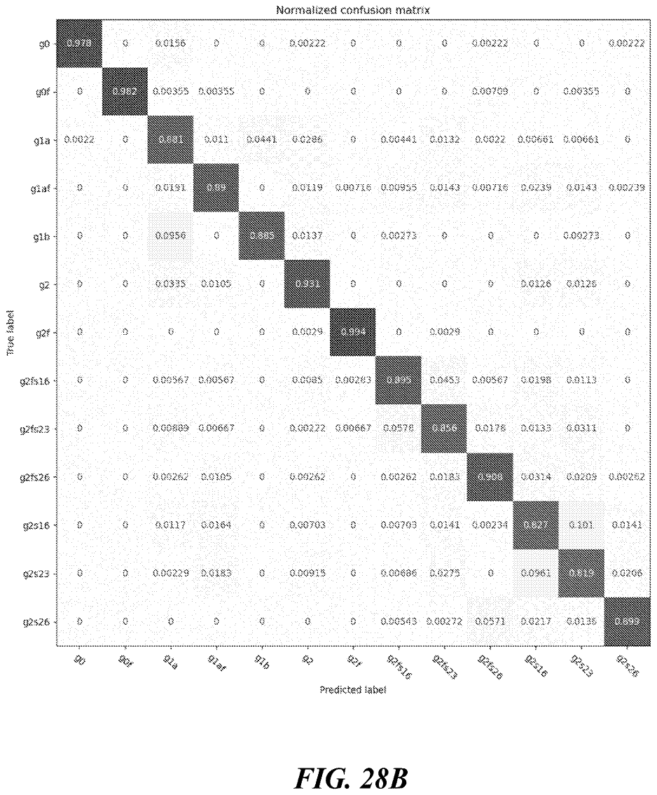

FIGS. 28A-28B show an example of a confusion matrix and associated Raman spectra, according to some embodiments. FIG. 28A shows a plurality of Raman spectra of various glycans as wild types, fucosylated, isomers, and with glycosidic linkages. The Raman spectra of FIG. 28A were processed using the clustering algorithms of the present disclosure, resulting in the confusion matrix of FIG. 28B. The confusion matrix shows highly accurate categorization of the glycans, showing the utility of the methods and systems of the present disclosure in glycan profiling.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

What is claimed is:

1. A method of determining an identity of an analyte, comprising:
(a) providing a system comprising:
(i) chip comprising a resonator comprising a nanogap, wherein said nanogap comprises said analyte,
(ii) a light source, and
(iii) a detector;
(b) illuminating said resonator using light from said light source, wherein said resonator concentrates a field of said light into said nanogap;
(c) detecting, using said detector, an emission light generated by an interaction of said field with said analyte; and
(d) processing said emission light or a derivative there of to determine said identity of said analyte.

2. The method of claim 1, wherein said resonator comprises an array of non-uniform features.

3. The method of claim 2, wherein a feature of said array of non-uniform features comprises said nanogap.

4. The method of claim 1, wherein (d) comprises using said emission light to generate a spectrum, and processing said spectrum to determine said identity.

5. The method of claim 1, wherein said interaction is a Raman interaction.

6. The method of claim 1, wherein said analyte is a biological analyte.

7. The method of claim 6, wherein said biological analyte is a protein, polypeptide, or peptide.

8. The method of claim 1, wherein said analyte is a chemical analyte.

9. The method of claim 1, wherein said analyte does not comprise a label or have a label bound thereto.

10. The method of claim 1, wherein said light source is a laser light source.

11. A chip for analyzing a biological sample, said chip comprising an array of non-uniform features, wherein a feature of said array of non-uniform features comprises an electrical insulator or a semiconductor, wherein said feature comprises a nanogap.

12. The chip of claim 1, wherein said nanogap is configured to concentrate an incident light.

13. The chip of claim 12, wherein said incident light is an incident laser.

14. The chip of claim 13, wherein said incident laser is at a wavelength of at least about 400 nanometers (nm) to at least about 1800 nanometers (nm).

15. The chip of claim 12, wherein said incident light is a light emitting diode (LED) light.

16. The chip of claim 12, wherein said incident light is a lamp.

17. The chip of claim 1, wherein said nanogap comprises a binding moiety that is specific or non-specific for said analyte.

18. The chip of claim 1, wherein said nanogap comprises a binding moiety with binding specificity for said analyte.

19. The chip of claim 1, wherein said biological sample comprises two or more components.

20. The chip of claim 19, further comprising an additional array comprising non-uniform features configured to filter said two or more components according to size, charge, or binding affinity.

21. The chip of claim 20, wherein said non-uniform pillars of said additional array are interspersed with a plurality of electrodes or functionalized features configured to filter said two or more components according to charge or size.

22. The chip of claim 21, wherein said functionalized feature comprises a functionalized oxide surface.

23. The chip of claim 1, wherein said biological sample is a tissue sample.

24. The chip of claim 1, wherein said biological sample is a single cell.

25. The chip of claim 1, wherein said analyte is a polynucleotide.

26. The chip of claim 1, wherein said analyte is a protein, polypeptide, or peptide.

27. The chip of claim 1, wherein said analyte is a metabolite.

28. The chip of claim 1, wherein said biological sample is an organism.

29. The chip of claim 28, wherein said organism is a bacterium.

30. The chip of claim 28, wherein said organism is a virus.

31. The chip of claim 1, wherein said biological sample comprises a cell fragment.

32. The chip of claim 1, wherein at least one feature of said array has a height of at least about 50 nanometers (nm) to at least about 1000 nanometers (nm).

33. The chip of claim 1, wherein at least one feature of said array has a width of at least about 50 nanometers (nm) to at least about 500 nanometers (nm).

34. The chip of claim 1, wherein at least one feature of said array has a length of at least about 50 nanometers (nm) to at least about 2000 nanometers (nm).

35. The chip of claim 1, wherein the distance between said non-uniform features is about 50 nanometers (nm) to about 1000 nanometers (nm).

36. The chip of claim 1, further comprising a first subset of said array of non-uniform features and a second subset of said array of non-uniform features, wherein said first subset and said second subset are adjacent to one another.

37. The chip of claim 36, wherein said first subset of said array of non-uniform features is separated from said second subset of said array of non-uniform features by a distance of at least about 5 nanometers (nm) to at least about 3000 nanometers (nm).

38. The chip of claim 36, wherein said first subset of said array of non-uniform features is separated from said second subset of said array of non-uniform features by a distance of at least about 2000 nanometers (nm).

39. The chip of claim 36, wherein said first subset of said array of non-uniform features is separated from said second subset of said array of non-uniform features by a distance of less than about 1000 nanometers (nm).

40. The chip of claim 36, wherein said first subset of said array of non-uniform features is parallel to said second subset of said array of non-uniform features.

41. The chip of claim 40, wherein said first subset of said array of non-uniform features is separated from said second subset of said array of non-uniform features by one or more dielectric fins.

42. The chip of claim 36, wherein said first subset of said array of non-uniform features is not parallel to said second subset of said array of non-uniform features.

43. The chip of claim 1, wherein two or more non-uniform features of said array comprise a nanogap.

44. The chip of claim 1, wherein three or more non-uniform features of said array comprise a nanogap.

45. The chip of claim 1, wherein each said feature of said array comprises a nanogap.

46. The chip of claim 1, wherein said nanogap is at least about 5 nanometers (nm) to at most about 150 nm wide.

47. The chip of claim 46, wherein said nanogap is at least about 5 nm to at most about 70 nm wide.

48. The chip of claim 47, wherein said nanogap is at least about 5 nm to at most about 30 nm wide.

49. The chip of claim 1, wherein said non-uniform features of said array comprise one or more materials from the group consisting of silicon, silicon nitride, aluminum nitride, titanium dioxide, silicon dioxide, gallium nitride, hafnium oxide, germanium, and silicon carbide.

50. The chip of claim 1, wherein said chip is provided on a substrate.

51. The chip of claim 50, wherein said substrate comprises one or more materials from the group consisting of germanium aluminum oxide, silicon dioxide, fused silica, silicon dioxide on silicon, silicon, silicon nitride, gallium nitride, calcium fluoride, and beryllium oxide.

52. The chip of claim 1, wherein two or more of said non-uniform features of said array are parallel to one another.

53. The chip of claim 1, wherein two or more of said non-uniform features of said array are nonparallel to one another.

54. The chip of claim 1, wherein said non-uniform features of said array are rectangular.

55. The chip of claim 1, wherein said non-uniform features of array are rounded.

56. The chip of claim 1, wherein said non-uniform features of said array are arranged in a periodic configuration.

57. The chip of claim 1, wherein said non-uniform features of said array are arranged in a nonperiodic configuration.

58. The chip of claim 1, wherein a non-uniform feature of said array is a photonic crystal mirror.

59. The chip of claim 1, wherein said array has a quality factor of at least about 100.

60. The chip of claim 1, wherein said array has a mode volume of at most about 800 nanometers.

61. A method of detecting or identifying an analyte in a biological sample, comprising:
(a) providing said biological sample on a chip comprising a resonator comprising a nanogap;
(b) exposing said chip to a first light from a light source, such that said first light interacts with said resonator and is further concentrated in said nanogap;
(c) detecting a second light from said resonator subsequent to said resonator being exposed to said first light; and
(d) using said second light to detect or identify said analyte.

62. The method of claim 61, wherein said resonator comprises an array comprising a plurality of non-uniform features, wherein a feature of said plurality of non-uniform features comprises said nanogap.

63. The method of claim 61, wherein said second light yields infrared scattering signature associated with said analyte.

64. The method of claim 61, wherein said second light yields a vibrational scattering signature associated with said analyte.

65. The method of claim 64, wherein said vibrational scattering signature is a Raman spectrum.

66. The method of claim 61, wherein said second light yields autofluorescence associated with said analyte.

67. The method of claim 61, wherein said light source of said first light is integrated with said chip.

68. The method of claim 61, wherein said light source of said first light is not integrated with said chip.

69. The method of claim 61, wherein said a light source of said second light is integrated with said chip.

70. The method of claim 61, wherein said light source of said second light is not integrated with said chip.

71. The method of claim 61, further comprising detecting the presence or absence of said analyte.

72. The method of claim 61, wherein said nanogap comprises a binding moiety with binding specificity for said analyte.

73. The method of claim 61, wherein said nanogap comprises a binding moiety with binding specificity for said analyte.

74. The method of claim 61, wherein said nanogap is configured to concentrate an incident light.

75. The method of claim 61, wherein said first light is an incident laser.

76. The method of claim 75, wherein said incident laser is at a wavelength of at least about 400 nanometers (nm) to at least about 1800 nanometers (nm).

77. The method of claim 61, wherein said first light is a light emitting diode (LED) light.

78. The method of claim 61, wherein said first light is a lamp.

79. The method of claim 61, further comprising providing a detector.

80. The method of claim 79, further comprising using said detector to scan wavelengths in a detector plane.

81. The method of claim 61, further comprising using super resolution imaging to image at least a portion of said chip.

82. The method of claim 81, wherein said super resolution imaging is structured illumination microscopy (SIM).

83. The method of claim 81, wherein said super resolution imaging is entropy based super resolution imaging (ESI).

84. The method of claim 81, wherein said super resolution imaging is stochastic optical reconstruction microscopy (STORM).

85. The method of claim 81, wherein said super resolution imaging is super resolution optical fluctuation imaging (SOFI).

86. The method of claim 81, wherein said super resolution imaging is stimulated emission depletion microscopy (STED).

87. The method of claim 81, further comprising producing one or more hyperspectral images.

88. The method of claim 87, wherein each of said one or more hyperspectral images represents a distinct Raman signature.

89. The method of claim 87, further comprising collecting data from said one or more hyperspectral images.

90. The method of claim 61, further comprising developing a machine learning model.

91. The method of claim 90, wherein a computing system is configured to execute said machine learning model.

92. The method of claim 91, wherein said computing system comprises a neural network.

93. The method of claim 92, wherein said neural network is a convolutional neural network (CNN).

94. The method of claim 61, wherein said biological sample comprises two or more components.

95. The method of claim 94, further comprising an additional array of non-uniform features configured to filter said two or more components according to size, charge, or binding affinity.

96. The method of claim 95, wherein said additional array of non-uniform features is interspersed with a plurality of electrodes or functionalized features configured to filter said two or more components according to charge.

97. The method of claim 96, wherein said functionalized feature comprises a functionalized oxide surface.

98. The method of claim 61, wherein said biological sample is a tissue sample.

99. The method of claim 61, wherein said biological sample is a single cell.

100. The method of claim 61, wherein said analyte is a polynucleotide.

101. The method of claim 61, wherein said analyte is a protein, polypeptide, or peptide.

102. The method of claim 61, wherein said analyte is a metabolite.

103. The method of claim 61, wherein said biological sample is an organism.

104. The method of claim 103, wherein said organism is a bacterium.

105. The method of claim 103, wherein said organism is a virus.

106. The method of claim 61, wherein said biological sample comprises a cell fragment.

107. The method of claim 61, wherein at least one feature of said resonator has a height of at least about 50 nanometers (nm) to at least about 1000 nanometers (nm).

108. The method of claim 61, wherein at least one feature of said resonator has a width of at least about 50 nanometers (nm) to at least about 500 nanometers (nm).

109. The method of claim 61, wherein at least one feature of said resonator has a length of at least about 50 nanometers (nm) to at least about 2000 nanometers (nm).

110. The method of claim 62, wherein the distance between said non-uniform features of said array is about 50 nanometers (nm) to about 1000 nanometers (nm).

111. The method of claim 62, further comprising a first subset of said array of non-uniform features and a second subset of said array of non-uniform features, wherein said first subset and said second subset are adjacent to one another.

112. The method of claim 111, wherein said first subset of said array of non-uniform features is separated from said second subset of said array of non-uniform features by a distance of at least about 5 nanometers (nm) to at most about 3000 nanometers (nm).

113. The method of claim 111, wherein said first subset of said array of non-uniform features is separated from said second subset of said array of non-uniform features by a distance of at least about 2000 nanometers (nm).

114. The method of claim 111, wherein said first subset of said array of non-uniform features is separated from said second subset of said array of non-uniform features by a distance of less than about 1000 nanometers (nm).

115. The method of claim 111, wherein said first subset of said array of non-uniform features is parallel to said second subset of said array of non-uniform features.

116. The method of claim 115, wherein said first subset of said array of non-uniform features is separated from said second subset of said array of non-uniform features by one or more dielectric fins.

117. The method of claim 111, wherein said first subset of said array of non-uniform features is not parallel to said second subset of said array of non-uniform features.

118. The method of claim 61, wherein two or more non-uniform features of said resonator comprise a nanogap.

119. The method of claim 61, wherein three or more non-uniform features of said resonator comprise a nanogap.

120. The method of claim 61, wherein each said feature of said resonator comprises a nanogap.

121. The method of claim 61, wherein said nanogap is at least about 5 nanometers (nm) to at most about 150 nm wide.

122. The method of claim 121, wherein said nanogap is at least about 5 nm to at most about 70 nm wide.

123. The method of claim 122, wherein said nanogap is at least about 5 nm to at most about 30 nm wide.

124. The method of claim 62, wherein said non-uniform features of said array comprise one or more materials from the group consisting of silicon, silicon nitride, aluminum nitride, titanium dioxide, silicon dioxide, gallium nitride, hafnium oxide, germanium, and silicon carbide.

125. The method of claim 61, wherein said chip is provided on a substrate.

126. The method of claim 61, wherein said substrate comprises one or more materials from the group consisting of germanium, aluminum oxide, silicon dioxide, fused silica, silicon dioxide on silicon, silicon, silicon nitride, gallium nitride, calcium fluoride, and beryllium oxide.

127. The method of claim 62, wherein two or more of said non-uniform features of said array are parallel to one another.

128. The method of claim 62, wherein two or more of said non-uniform features of said array are nonparallel to one another.

129. The method of claim 62, wherein said non-uniform features of said array are rectangular.

130. The method of claim 62, wherein said non-uniform features of said array are rounded.

131. The method of claim 62, wherein said non-uniform features of said array are arranged in a periodic configuration.

132. The method of claim 62, wherein said non-uniform features of said array are arranged in a nonperiodic configuration.

133. The method of claim 62, wherein a non-uniform feature of said array is a photonic crystal mirror.

134. A label-free method for detecting an analyte, comprising providing a chip comprising said analyte, wherein said analyte is label-free, and using light to detect an identity of said analyte at a sensitivity of at least 80%, a specificity of at least 80%, or an accuracy of at least 80%.
